SEQUENCE LISTING (2 sequences)

SEQ ID NO: 1 - protein, 1091 aa, Human
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQGQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPGEWLVWKQSCSSTSSTHSAAASLQCPSQVLPPASPEGETVADLQTQ

SEQ ID NO: 2 - DNA, 3276 bp, Human
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacagggacaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctggtgagtggcttgtctggaaacagtcctgctcctcaacctcctcgacccactcagcagcagccagtctccagtgtccaagccaggtgctccctccagcatctccagagggggaaacagtggcagatttgcagacacagtga

(19) United States
(12) Patent Application Publication
(10) Pub. No.: US 2010/0248249 A1
(43) Pub. Date: Sep. 30, 2010

(54) Methods for Assessing Cancer for Increased Sensitivity to 10-Propargyl-10-Deazaaminopterin by Assessing Egfr Levels

(75) Inventor: Gijsbertus J. Pronk, Arvada, CO (US)

Correspondence Address: SWANSON & BRATSCHUN, L.L.C., 8210 SOUTHPARK TERRACE, LITTLETON, CO, 80120, US

(73) Assignee: ALLOS THERAPEUTICS, INC., Westminster, CO (US)

(21) Appl. No.: 12/779,852

(22) Filed: May 13, 2010

Related U.S. Application Data
(63) Continuation-in-part of application No. 12/193,518, filed on Aug. 18, 2008, now abandoned.
(60) Provisional application No. 60/956,525, filed on Aug. 17, 2007.
(60) Provisional application No. 61/044,823, filed on Apr. 14, 2008.

(51) Int. Cl. C12Q 1/68; C12Q 1/02

(52) U.S. Cl. 435/6; 435/29

(57) ABSTRACT

The present invention relates to a method for assessing the sensitivity of a patient's cancer to treatment with 10-propargyl-10-deazaminopterin and a method for selecting a patient for treatment of cancer with 10-propargyl-10-deazaminopterin, by determining the amount of a EGFR or other growth factor expressed by the cancer and comparing the amount with the amount of EGFR or other growth factor expressed by a reference cancer.

### RELATED APPLICATIONS

The instant application claims priority to and is a continuation in part of U.S. Ser. No. 12/193,518, filed Aug. 18, 2008, entitled “Combination of 10-Propargyl-10-Deazaminopterin and Erlotinib for the Treatment of Non-Small Cell Lung Cancer,” which claims priority to U.S. Ser. No. 60/956,525, filed Aug. 17, 2007, entitled “Combination of 10-Propargyl-10-Deazaminopterin and Erlotinib for the Treatment of Non-Small Cell Lung Cancer,” and to U.S. Ser. No. 61/044,823, filed Apr. 14, 2008, entitled “Combination of 10-Propargyl-10-Deazaminopterin and Erlotinib for the Treatment of Non-Small Cell Lung Cancer,” each of which is incorporated herein in their entirety by reference for all that they teach and disclose.

### TECHNICAL FIELD

The present invention relates to methods to treat cancer with 10-propargyl-10-deazaminopterin and methods for assessing cancers and selecting patients for treatment based for increased sensitivity to 10-propargyl-10-deazaminopterin. The present invention also relates to methods to treat non-small cell lung cancer with combinations of 10-propargyl-10-deazaminopterin and an EGFR Kinase inhibitor, including erlotinib.

### SEQUENCE INCORPORATED BY REFERENCE

Incorporated by reference herein in its entirety is the Sequence Listing co-submitted with the instant application, entitled “Sequence_listing_ST25.txt”, created May 13, 2010, size of 14 kilobytes.

### BACKGROUND OF THE INVENTION

The epidermal growth factor receptor (EGFR; also known as ErbB-1; HER1) is the cell-surface receptor for members of the epidermal growth factor family (EGF-family) of extracellular protein ligands. The epidermal growth factor receptor is a member of the ErbB family of receptors, a subfamily of four closely related receptor tyrosine kinases: EGFR (ErbB-1), HER2/c-neu (ErbB-2), Her 3 (ErbB-3) and Her 4 (ErbB-4). ErbB receptors bind EGF-like growth factors, such as EGF and neuregulins (NRGs), and mediate multiple cellular responses, such as proliferation, differentiation, migration, and survival. Aberrations in ErbB signaling have a role in carcinogenesis, and cancer drugs targeting EGFR and ErbB2 are currently in clinical use.

EGFR exists on the cell surface and is activated by binding of its specific ligands, including epidermal growth factor and transforming growth factor α (TGFα). ErbB2 has no known direct activating ligand, and may be in an activated state constitutively or become active upon heterodimerization with other family members such as EGFR.

Upon activation by its growth factor ligands, EGFR undergoes a transition from an inactive monomeric form to an active homodimer—although there is some evidence that preformed inactive dimers may also exist before ligand binding. In addition to forming homodimers after ligand binding, EGFR may pair with another member of the ErbB receptor family, such as ErbB2/Her2/neu, to create an activated heterodimer. There is also evidence to suggest that clusters of activated EGFRs form, although it remains unclear whether this clustering is important for activation itself or occurs subsequent to activation of individual dimers.

EGFR dimerization stimulates its intrinsic intracellular protein-tyrosine kinase activity. As a result, autophosphorylation of several tyrosine (Y) residues in the C-terminal domain of EGFR occurs. These include Y992, Y1045, Y1068, Y1148 and Y1173. This autophosphorylation elicits downstream activation and signaling by several other proteins that associate with the phosphorylated tyrosines through their own phosphotyrosine-binding SH2 domains. These downstream signaling proteins initiate several signal transduction cascades, principally the MAPK, Akt and JNK pathways, leading to DNA synthesis and cell proliferation. Such proteins modulate phenotypes such as cell migration, adhesion, and proliferation. Activation of the receptor is important for the innate immune response in human skin. The kinase domain of EGFR can also cross-phosphorylate tyrosine residues of other receptors it is aggregated with, and can itself be activated in that manner.

10-Propargyl-10-deazaminopterin (encompassing “10-propargyl-10-dAM,” “pralatrexate,” “racemic PDX,” “(2S)-2-[[4-[(1RS)-1-[(2,4-diaminopteridin-6-yl)methyl]but-3-ynyl]benzoyl]amino]pentanedioic acid,” “(2RS)-2-[[4-[(1RS)-1-[(2,4-diaminopteridin-6-yl)methyl]but-3-ynyl]benzoyl]amino]pentanedioic acid,” and “PDX”), is a compound which has been tested and found useful in the treatment of cancer. 10-propargyl-10-deazaminopterin has been approved by the U.S. Food and Drug Administration (FDA) as a treatment for relapsed and refractory peripheral T-cell lymphoma. 10-propargyl-10-deazaminopterin is also being investigated for use in lymphoma, lung cancer, bladder cancer, and breast cancer.

10-propargyl-10-deazaminopterin was originally disclosed by DeGraw et al., “Synthesis and Antitumor Activity of 10-Propargyl-10-deazaminopterin,” J. Med. Chem. 36: 2228-2231 (1993) and shown to act as an inhibitor of the enzyme dihydrofolate reductase (“DHFR”) and as an inhibitor of growth in the murine L1210 cell line. In addition, some results were presented for the antitumor properties of the compound using the E0771 murine mammary tumor model.

U.S. Pat. No. 6,028,071 and PCT Publication No. WO 1998/02163, disclose that highly purified 10-propargyl-10-deazaminopterin compositions when tested in a xenograft model have efficacy against human tumors. Subsequent studies with 10-propargyl-10-deazaminopterin have shown that it is useful on its own and in combinations with other therapeutic agents. For example, Sirotnak et al., Clinical Cancer Research Vol. 6, 3705-3712 (2000) reports that co-administration of 10-propargyl-10-deazaminopterin and probenecid, an inhibitor of a cMOAT/MRP-like plasma membrane ATPase, greatly enhances the efficacy of 10-propargyl-10-deazaminopterin against human solid tumors. 10-propargyl-10-deazaminopterin and combinations of 10-propargyl-10-deazaminopterin with platinum based chemotherapeutic agents have been shown to be effective against mesothelioma. (Khokar, et al., Clin. Cancer Res. 7: 3199-3205 (2001). Co-administration with gemcitabine (Gem), for treatment of lymphoma, has been disclosed in WO/2005/117892. Combinations of 10-propargyl-10-deazaminopterin with taxols are disclosed to be efficacious in U.S. Pat. No. 6,323,205. 10-propargyl-10-deazaminopterin has also shown to be effective for treatment of T-cell lymphoma, see U.S. Pat. No. 7,622,470. Other studies have shown a method for assessing sensitivity of a lymphoma to treatment with 10-propargyl-10-deazaminopterin by determining the amount of reduced folate carrier-1 protein (RFC-1) expressed by the sample, wherein a higher level of expressed RFC-1 is indicative of greater sensitivity to 10-propargyl-10-deazaminopterin, disclosed in PCT Publication No. WO 2005/117892.

10-propargyl-10-deazaminopterin is known as an antifolate/antimetabolite. Several proteins are implicated in the metabolism of folic acid and as targets of anti-folates such as 10-propargyl-10-deazaminopterin and methotrexate (MTX) in tumor cells. In most tumor cells, RFC-1 mediates internalization of folate analogs. Once inside the cell, these analogs either bind dihydrofolate reductase (DHFR), thereby depleting intracellular reduced folate pools needed for purine and thymidine biosynthesis, or will be metabolized to a polyglutamates prior to binding to DHFR. Polyglutamylation is catalyzed by folyl-polyglutamate synthetase (FPGS). Folyl-poly glutamate hydrolase (FPGH, also known as gamma-glutamyl hydrolase [GGH]) mediates cleavage and thus subsequent clearance of these intracellular polyglutamated anti-folates. Thymidylate synthase (TS) and glycinamide ribonucleotide formyl transferase (GARFT) are also involved in folate metabolism as “recycling” enzymes (thus directly affecting pools of nucleotides available for DNA synthesis). Without intending to be bound by a specific mechanism, it is believed that the correlation between RFC-1 expression levels and 10-propargyl-10-deazaminopterin sensitivity is a reflection of increased transport of 10-propargyl-10-deazaminopterin into tumor cells. Without being bound by theory, it is believed that alterations in other folate pathway enzymes discussed herein also correlate with 10-propargyl-10-deazaminopterin sensitivity; such as, for example, reduced DHFR levels correlating with a decrease in the amount of intracellular drug required to inhibit this enzyme, reduced GARFT and TS potentially reducing the pools of available nucleotides, increased FPGS increasing the rate of polyglutamylation of 10-propargyl-10-deazaminopterin and resulting in increased retention within the cell to facilitate ongoing activity against DHFR.

One of the continued problems with therapy in cancer patients is individual differences in response to therapies. While advances in development of successful cancer therapies progress, only a subset of patients respond to any particular therapy. With the narrow therapeutic index and the toxic potential of many available cancer therapies, such differential responses potentially contribute to patients undergoing unnecessary, ineffective and even potentially harmful therapy regimens. If a designed therapy could be optimized to treat individual patients, such situations could be reduced or even eliminated. Furthermore, targeted designed therapy may provide more focused, successful patient therapy overall. Accordingly, there is a need to identify particular cancer patients who are expected to have a favorable outcome when administered particular cancer therapies as well as particular cancer patients who may have a favorable outcome using more aggressive and/or alternative cancer therapies, e.g., alternative to previous cancer therapies administered to the patient. It would therefore be beneficial to provide for the diagnosis, staging, prognosis, and monitoring of cancer patients, including, e.g., hematological cancer patients (e.g., multiple myeloma, leukemias, lymphoma, etc.) who would benefit from particular cancer inhibition therapies as well as those who would benefit from a more aggressive and/or alternative cancer inhibition therapy, e.g., alternative to a cancer therapy or therapies the patient has received, thus resulting in appropriate preventative measures.

### SUMMARY OF THE INVENTION

The present invention relates to a method for assessing the sensitivity of a patient's cancer to treatment with 10-propargyl-10-deazaminopterin and a method for selecting a patient for treatment of cancer with 10-propargyl-10-deazaminopterin, by determining the amount of a selected polypeptide expressed by the cancer and comparing the amount with the amount of the selected polypeptide expressed by a reference cancer, wherein the polypeptide includes a member of a growth factor pathway within cells which includes epidermal growth factor receptor (EGFR).

In one embodiment, the present invention includes a method of selecting a patient for treatment of a cancer with 10-propargyl-10-deazaminopterin. The method can comprise the following steps, in any order. One step comprises obtaining a sample of the patient's cancer tissue; another step comprises determining the expression level of epidermal growth factor receptor (EGFR) expressed in the sample; another step includes comparing the determined expression level in the sample with a reference expression level for EGFR; and another step includes selecting the patient for treatment 10-propargyl-10-deazaminopterin where the comparison of the expression level in the sample of EGFR and the corresponding reference expression level indicate sensitivity of patient's cancer tissue to 10-propargyl-10-deazaminopterin. In some embodiments, the patient's cancer is a solid tumor. In some embodiments, the solid tumor can be any of the following: NSCLC, head and neck cancer, prostate cancer, and breast cancer.

In another embodiment, the present invention includes a method for assessing sensitivity of a patient's cancer to treatment with 10-propargyl-10-deazaminopterin. This method can include the following steps, in any order. One step includes obtaining a sample of the patient's cancer tissue. Another step includes determining the expression level of at EGFR expressed in the sample. Another step includes comparing the determined expression level in the sample with a reference expression level for EGFR to determine whether the expression level for EGFR in the sample is indicative of an expression level that predicts sensitivity to 10-propargyl-10-deazaminopterin. Another step includes generating a report of the predicted sensitivity of the sample to 10-propargyl-10-deazaminopterin. In some embodiments, the patient's cancer is a solid tumor. In some embodiments, the solid tumor can be any of the following: NSCLC, head and neck cancer, prostate cancer, and breast cancer.

In another embodiment, the present invention includes a method for assessing sensitivity of a cancer to treatment with 10-propargyl-10-deazaminopterin. This method includes the following steps, in any order. One step includes obtaining a sample of the lymphoma. Another step includes determining the amount of EGFR expressed by the sample wherein higher levels of expressed EGFR are indicative of sensitivity to 10-propargyl-10-deazaminopterin; and generating a report of the predicted sensitivity of the sample to 10-propargyl-10-deazaminopterin.

### BRIEF DESCRIPTION OF THE DRAWINGS

FIG. 1 shows a synthetic scheme useful in preparing 10-propargyl-10-deazaminopterin;

FIG. 2 shows sensitivity of PDX and other folate inhibitors to 15 cancer cell lines tested;

FIG. 3 shows the relative mRNA expression (quantitative RT-PCR) of genes implicated in proliferation, apoptosis and cell signaling in 10-propargyl-10-deazaminopterin-sensitive and 10-propargyl-10-deazaminopterin-resistant groups of cell lines;

FIG. 4 shows effects of 10-propargyl-10-deazaminopterin and methotrexate exposure on phosphorylation of several intracellular kinases; phosphorylation of b-Raf, MEK, ERK, and c-JUN was assessed using Western blot;

FIG. 5 shows effects of 10-propargyl-10-deazaminopterin-erlotinib combination on phosphorylation of ERK 1/2.

FIGS. 6A and 6B show the combinations of pralatrexate with erlotinib or lapatinib was synergistic when pralatrexate was given first or simultaneously with these EGFR kinase inhibitors.

FIGS. 7A and 7B show the combinations of pralatrexate with erlotinib or lapatinib was synergistic when pralatrexate was given first or simultaneously with these EGFR kinase inhibitors.

### DETAILED DESCRIPTION OF THE INVENTION

One of the continued problems with therapy in cancer patients is individual differences in response to therapies. While advances in development of successful cancer therapies progress, only a subset of patients respond to any particular therapy. With the narrow therapeutic index and the toxic potential of many available cancer therapies, such differential responses potentially contribute to patients undergoing unnecessary, ineffective and even potentially harmful therapy regimens. If a designed therapy could be optimized to treat individual patients, such situations could be reduced or even eliminated. Furthermore, targeted designed therapy may provide more focused, successful patient therapy overall. Accordingly, there is a need to identify particular cancer patients who are expected to have a favorable outcome when administered particular cancer therapies as well as particular cancer patients who may have a favorable outcome using more aggressive and/or alternative cancer therapies, e.g., alternative to previous cancer therapies administered to the patient. It would therefore be beneficial to provide for the diagnosis, staging, prognosis, and monitoring of cancer patients, including, e.g., hematological cancer patients (e.g., multiple myeloma, leukemias, lymphoma, etc.) who would benefit from particular cancer inhibition therapies as well as those who would benefit from a more aggressive and/or alternative cancer inhibition therapy, e.g., alternative to a cancer therapy or therapies the patient has received, thus resulting in appropriate preventative measures.

A cancer is “responsive” to a therapeutic agent or there is a “good response” to a treatment if its rate of growth is inhibited as a result of contact with the therapeutic agent, compared to its growth in the absence of contact with the therapeutic agent. Growth of a cancer can be measured in a variety of ways, for instance, the size of a tumor or the expression of tumor markers appropriate for that tumor type may be measured. These criteria define the type of response measured and also the characterization of time to disease progression which is another important measure of a tumor's sensitivity to a therapeutic agent. The quality of being responsive 10-propargyl-10-deazaminopterin is a variable one, with different cancers exhibiting different levels of “responsiveness” to a given therapeutic agent, under different conditions. Still further, measures of responsiveness can be assessed using additional criteria beyond growth size of a tumor, including patient quality of life, degree of metastases, etc. In addition, clinical prognostic markers and variables can be assessed in applicable situations.

A cancer is “non-responsive” or has a “poor response” to a therapeutic agent such as 10-propargyl-10-deazaminopterin or there is a poor response to a treatment if its rate of growth is not inhibited, or inhibited to a very low degree, as a result of contact with the therapeutic agent when compared to its growth in the absence of contact with the therapeutic agent. As stated above, growth of a cancer can be measured in a variety of ways, for instance, the size of a tumor or the expression of tumor markers appropriate for that tumor type may be measured. The quality of being non-responsive to a therapeutic agent is a highly variable one, with different cancers exhibiting different levels of “non-responsiveness” to a given therapeutic agent, under different conditions. Still further, measures of non-responsiveness can be assessed using additional criteria beyond growth size of a tumor, including patient quality of life, degree of metastases, etc. In addition, clinical prognostic markers and variables can be assessed in applicable situations.

In one aspect, the present invention relates to a method for assessing the sensitivity of a patient's cancer to treatment with 10-propargyl-10-deazaminopterin and a method for selecting a patient for treatment of cancer with 10-propargyl-10-deazaminopterin, by determining the amount of a growth factor receptor polypeptide expressed by the cancer and comparing the amount with the amount of the growth factor receptor polypeptide expressed by a reference cancer. Sensitivity to 10-propargyl-10-deazaminopterin is indicated when the amounts of growth factor receptor polypeptide are comparable to, or greater than, the amount in a reference cancer, as discussed more fully below. The reference cancer can include a cancer that is sensitive to 10-propargyl-10-deazaminopterin.

“Treatment” can mean the use of a therapy to prevent or inhibit further tumor growth, as well as to cause shrinkage of a tumor, and to provide longer survival times. Treatment is also intended to include prevention of metastasis of tumor. A tumor is “inhibited” or “treated” if at least one symptom (as determined by responsiveness/non-responsiveness, time to progression, or indicators known in the art and described herein) of the cancer or tumor is alleviated, terminated, slowed, minimized, or prevented. Any amelioration of any symptom, physical or otherwise, of a tumor pursuant to treatment using a therapeutic regimen (e.g., 10-propargyl-10-deazaminopterin) as further described herein, is within the scope of the invention.

A growth factor receptor polypeptide of the present invention can include a member of the ErbB family of receptors, including EGFR (ErbB-1), HER2/c-neu (ErbB-2), Her 3 (ErbB-3) and Her 4 (ErbB-4). In one embodiment, the growth factor receptor polypeptide is epidermal growth factor receptor (EGFR.)

The fact that a growth factor receptor polypeptide, which includes EGFR, can act as such a “marker” or “biomarker” for activity for 10-propargyl-10-deazaminopterin is surprising, because 10-propargyl-10-deazaminopterin is known as an antifolate/antimetabolite with activity in at least one of the enzymes that are implicated in the metabolism of folic acid. By way of contrast, it is believed that 10-propargyl-10-deazaminopterin was not previously known to have significant interaction with growth factor or EGFR-mediated cell proliferation, cell differentiation, cell migration, and/or cell survival. Surprisingly, this application reports studies showing significant correlation between mRNA expression level and sensitivity to 10-propargyl-10-deazaminopterin, with nine sensitive human cancer cell lines having a higher average EGFR expression than the six resistant human cancer cell lines (p value=0.04), as well as increased activation of downstream kinases activated by EGFR upon treatment with 10-propargyl-10-deazaminopterin.

In one embodiment of the invention, the composition used for the methods of the instant invention can include 10-propargyl-10-deazaminopterin, including “highly purified” 10-propargyl-10-deazaminopterin, and diastereomers of 10-propargyl-10-deazaminopterin. As used in the specification and claims hereof, compositions which are “highly purified” contain 10-propargyl-10-deazaminopterin substantially free of other folic acid derivatives, particularly 10-deazaminopterin, which can interfere with the antitumor activity of the 10-propargyl-10-deazaminopterin. A composition within the scope of the invention may include carriers or excipients for formulating the 10-propargyl-10-deazaminopterin into a suitable dosage unit form for therapeutic use, as well as additional, non-folate therapeutic agents.

10-propargyl-10-deazaminopterin contains asymmetric centers at the carbon 10 (C10) and carbon 19 (C19) position. In one embodiment, 10-propargyl-10-deazaminopterin includes an approximately 1:1 racemic mixture of the R- and S-configurations at the C10 chiral center, and ≧98.0% of the S-diastereomer at the C19 chiral center. 10-propargyl-10-deazaminopterin includes the C10 diastereomers PDX-10a [S-configuration] Chemical name: (25)-2-[[4-[(1S)-1-[(2,4-diaminopteridin-6-yl)methyl]but-3-ynyl]benzoyl]amino]pentanedioic acid, and PDX-10b [R-configuration] Chemical name: (25)-2-[[4-[(1R)-1-[(2,4-diaminopteridin-6-yl)methyl]but-3-ynyl]benzoyl]amino]pentanedioic acid.

10-propargyl-10-deazaminopterin can be synthesized using the method disclosed in Example 7 of DeGraw et al., U.S. Pat. No. 5,354,751, which is directed to manufacturing 10-propargyl-10-deazaminopterin, is incorporated by reference herein in its entirety. 10-propargyl-10-deazaminopterin may also be synthesized by methods presented in U.S. Pat. No. 6,028,071, especially in Example 1, which example is incorporated by reference herein.

In order to generate diastereomers of 10-propargyl-10-deazaminopterin, 10-propargyl-10-deazaminopterin may be synthesized as taught herein and elsewhere, and either the final product or an earlier intermediate product may be subsequently used as a starting material to separate the C10 diastereomers. Alternately, a chiral synthesis may be employed where substantially pure PDX-10a and/or PDX-10b is produced directly from any of a number of starting materials. Chiral columns to separate enantiomers or diastereomers, known in the art, may be employed to separate the diastereomers of the final 10-propargyl-10-deazaminopterin or an earlier intermediate. Suitable chiral columns for separating the diastereomers include the chiral column CHIRALPAK AD, available from Daicel Chemical Industries Ltd., Japan, using ethanol as the mobile phase.

10-propargyl-10-deazaminopterin for use in a 10-propargyl-10-deazaminopterin-sensitive cancer according to the present invention will typically be administered to the patient in a dose regimen that provides for the most effective treatment (from both efficacy and safety perspectives) for which the patient is being treated, as known in the art. In conducting the treatment method of the present invention, the 10-propargyl-10-deazaminopterin for use in a 10-propargyl-10-deazaminopterin-sensitive cancer according to the present invention can be administered in any effective manner known in the art, such as by oral, topical, intravenous, intra-peritoneal, intramuscular, intra-articular, subcutaneous, intranasal, intra-ocular, vaginal, rectal, intracranial, or intradermal routes, depending upon the type of cancer being treated, and the medical judgment of the prescribing physician as based, e.g., on the results of published clinical studies.

10-propargyl-10-deazaminopterin for use in a 10-propargyl-10-deazaminopterin-sensitive cancer according to the present invention can be formulated as part of a pharmaceutical preparation. The specific dosage form will depend on the method of administration, but may include tablets, capsules, oral liquids, and injectable solutions for oral, intravenous, intramuscular, intracranial, or intraperitoneal administration, and the like. Dosing may be expressed as mg/m². Alternatively, dosing may be expressed as mg/kg body weight by any manner acceptable to one skilled in the art. One method for obtaining an equivalent dosing in mg/kg body weight involves applying the conversion factor 0.025 mg/kg, for an average human, as approximately equivalent to 1 mg/m². According to this calculation, dosing of 150 mg/m² is approximately equivalent to about 3.75 mg/kg.

Appropriate dosing for oncology for treatment of a 10-propargyl-10-deazaminopterin-sensitive cancer includes the following dosage regimes. For example, doses on the order of 10 to 120 mg/m² of body surface area/day (about 0.25 to 3 mg/kg body weight per day) are appropriate. Dosages of 30 mg/m² (about 0.75 mg/kg) once weekly for 3 weeks followed by a one week rest, 30 mg/m² (about 0.75 mg/kg) once weekly×6 weeks followed by a one week rest, or gradually increasing doses of 10-propargyl-10-deazaminopterin on the once weekly×6 week schedule are also suitable. Lower doses may be used as appropriate based on patient tolerance and type of malignancy. Higher doses can be utilized where less frequent administration is used. Thus, in a general sense, dosages of 10 to 275 mg/m² (about 0.25 to about 6.9 mg/kg) are suitably used with various dosing schedules, for example between about 100 to 275 mg/m² (about 2.5 to about 6.87 mg/kg) for biweekly dosages, and between about 10 to 150 mg/m² (about 0.25 to about 3.75 mg/kg), or, more specifically, between about 10 and 60 mg/m² for once weekly dosages.

The determination of suitable dosages using protocols similar to those described in U.S. Pat. No. 6,323,205 is within the skill in the art. In one embodiment, 10-propargyl-10-deazaminopterin for use in a 10-propargyl-10-deazaminopterin-sensitive cancer according to the present invention can be administered in an amount of from about 10 to about 275 mg/m² (about 0.25 to about 6.87 mg/kg) per dose. Methods of the present invention also include administration of 10-propargyl-10-deazaminopterin for use in a 10-propargyl-10-deazaminopterin-sensitive cancer according to the present invention weekly; in a dose of about 10 mg/m² (0.25 mg/kg) or about 30 mg/m² (0.75 mg/kg); in an amount of from about 10 to about 150 mg/m² (about 0.25 to about 3.75 mg/kg) per dose; biweekly; and in a dosage amount of about 100 to about 275 mg/m² (about 2.5 to about 6.9 mg/kg). In one embodiment, 10-propargyl-10-deazaminopterin for use in a 10-propargyl-10-deazaminopterin-sensitive cancer according to the present invention can be administered in an amount of between about 0.25 mg/kg and about 4 mg/kg; between about 0.75 mg/kg and about 3 mg/kg; in an amount between about 1.0 mg/kg and about 2.5 mg/kg; in an amount of about 0.25 mg/kg or about 0.75 mg/kg (or an equivalent amount in body surface area (BSA)).

10-propargyl-10-deazaminopterin may be used in combinations with other cytotoxic and antitumor compounds, including vinca alkaloids such as vinblastine, navelbine, and vindesine; probenicid, nucleotide analogs such as gemcitabine, 5-fluorouracil, and cytarabine; alkylating agents such as cyclophosphamide or ifosfamide; cisplatin or carboplatin; leucovorin; taxanes such a paclitaxel or docetaxel; anti-CD20 monoclonal antibodies, with or without radioisotopes, and antibiotics such as doxorubicin and mitomycin. Combinations of 10-propargyl-10-deazaminopterin with several of these other antitumor agents or with growth factor inhibitors and anti-angiogenic agents may also be used.

10-propargyl-10-deazaminopterin and other agents may be concurrently administered or utilized in combination as part of a common treatment regimen, in which the 10-propargyl-10-deazaminopterin and the other agent(s) are administered at different times. For example, the other agent may be administered before, immediately afterward or after a period of time (for example 24 hours) relative to the 10-propargyl-10-deazaminopterin administration. Thus, for purposes of this application, the term administering refers generally to concurrent administration or to sequential administration of the drugs and in either order in a parallel treatment regimen with or without a separation in time between the drugs unless otherwise specified.

10-propargyl-10-deazaminopterin is suitably used in combination with folic acid and vitamin B12 supplementation to reduce the side effects of the treatment. For example, patients may be treated with folic acid (1 mg/m² daily starting 1 week prior to treatment with 10-propargyl-10-deazaminopterin, or alternatively 1 mg perioral (p.o.) daily not based on body surface area (BSA)); and B12 (1 mg/m² monthly, or alternatively given intramuscularly (I.M.) every 8-10 weeks as 1 mg (not based on BSA), or alternatively p.o. daily 1 mg (not based on BSA)).

In one embodiment of the present invention, the invention includes a method for selecting a patient for treatment of a cancer with 10-propargyl-10-deazaminopterin, the method including the following steps (in any order.) One step includes obtaining a sample of the patient's cancer tissue. Another step includes determining the expression level of a growth factor receptor polypeptide. Another step includes comparing the determined expression level in the sample with a reference expression level for the same growth factor receptor polypeptide. In another step, the instant method includes selecting the patient for treatment with 10-propargyl-10-deazaminopterin, where the comparison of the expression level in the sample of the growth factor receptor polypeptide, and the corresponding reference expression level of the same, indicates or predicts sensitivity to 10-propargyl-10-deazaminopterin.

In another embodiment, the present invention includes a method for assessing the sensitivity of a patient's cancer to treatment with 10-propargyl-10-deazaminopterin. This method includes the following steps, in any order. One step includes obtaining a sample of the patient's cancer tissue. Another step includes determining the amount of a growth factor receptor polypeptide, expressed by the sample. Another step optionally includes obtaining a reference expression level for the same growth factor receptor polypeptide, for a cancer having sensitivity to 10-propargyl-10-deazaminopterin. The sample cancer and the reference cancer may be the same or different. Another step includes comparing the determined expression level in the sample with a reference expression level for the growth factor receptor polypeptide, to determine whether the expression level for the growth factor receptor polypeptide in the sample is a match to, is similar to, is greater than, or is otherwise indicative of an expression level that predicts sensitivity to 10-propargyl-10-deazaminopterin. Another optional step includes the step of generating a report of predicted sensitivity of the sample to 10-propargyl-10-deazaminopterin.

In one embodiment of the invention, an expression level of a growth factor receptor polypeptide that predicts sensitivity to 10-propargyl-10-deazaminopterin is one that “matches,” or is similar to, is greater than, or in an amount that otherwise indicates sensitivity, the level of the same growth factor receptor polypeptide of a 10-propargyl-10-deazaminopterin sensitive cancer. The cancer type for the reference may be the same or different than the cancer type for the sample, but in one embodiment is the same type of cancer.

In one embodiment, a “match,” or a level that is similar to, is greater than, or in an amount that otherwise indicates sensitivity of the sample expression level of a growth factor receptor polypeptide indicates the patient's cancer has increased sensitivity to 10-propargyl-10-deazaminopterin.

Another step includes generating a report of the sensitivity of the sample to 10-propargyl-10-deazaminopterin. A report may be, without limitation, an oral report, a printed report, or an electronically transmitted report.

A growth factor receptor polypeptide, which includes EGFR, is also variously referred to herein as a “biomarker of the invention,” “biomarker,” “marker,” “peptide,” “selected peptide,” or a plural thereof, and the like.

In another embodiment of the present invention, a method of selecting a patient for treatment of a cancer with 10-propargyl-10-deazaminopterin is provided. The method includes the following steps, in any order. One step includes obtaining a sample of the patient's cancer tissue. Another step includes determining the amount of at least one growth factor receptor polypeptide expressed by the sample. Another optional step includes obtaining a reference expression level for the at least one growth factor receptor pathway polypeptide for a cancer having sensitivity to 10-propargyl-10-deazaminopterin. Another step includes comparing the expression data for the growth factor receptor polypeptide with the reference expression for the same growth factor receptor polypeptide.

In some steps of the methods of the present invention, the level of expression of the RNA and/or protein products of one or more growth factor receptor polypeptides of the invention, as measured by the amount or level of RNA or protein, is compared to see if the level of expression is similar to, a match to, or if the sample has significantly greater expression than the reference, or the respective levels is otherwise indicative of sensitivity, to a person of skill in the art. The term “match” indicates that the level of expression of protein or mRNA, and/or one or more spliced variants of mRNA of the biomarker in the sample is compared with the level of expression of the same one or more biomarkers of the invention as measured by the amount or level of protein, or level of RNA, including mRNA and/or one or more spliced variants of mRNA in a reference sample, and is determined to be similar to, a match to, or the sample has significantly greater expression than the reference, or the respective levels are otherwise indicative of sensitivity, to a person of skill in the art and/or in accordance with the discussion herein below.

Determining if the sample is similar to, a match to, a reference, or the sample has significantly greater expression than the reference, or the respective levels are otherwise indicative of sensitivity, to a person of skill in the art, can also include a measurement of the protein, or one or more protein variants encoded by a growth factor receptor polypeptide of the invention in the sample as compared with the amount or level of protein expression, including one or more protein variants of the same growth factor receptor polypeptide s of the invention in the reference sample.

Similarity to, a match to, or wherein the sample has significantly greater expression than the reference, or the respective levels is otherwise indicative of sensitivity, to a person of skill in the art includes a level of expression (mRNA or protein) in the sample that is at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 98%, at least about 100%, at least about 110%, at least about 120%, at least about 130%, at least about 140%, at least about 150%, at least about 160%, at least about 170%, at least about 180%, at least about 200%, at least about three fold, at least about four fold, at least about five fold, at least about ten-fold, of the reference.

In some embodiments, similarity to, or a match with, the reference level is an amount of the growth factor receptor polypeptide in the sample is an amount that is significantly greater than the amount of the growth factor receptor polypeptide in the reference. In those embodiments, the amount of the selected growth factor receptor polypeptide may be significantly enhanced over the reference expression level, such as, ten-fold greater, twenty-fold greater, fifty-fold greater, one hundred-fold greater or more.

A “normal” amount of a marker may refer to the amount of a “reference sample”, (e.g., sample from a healthy subject not having the marker-associated disease), preferably, the average expression level of the marker in several healthy subjects. A reference sample amount may be comprised of an amount of one or more markers from a reference database. Alternatively, a “normal” level of expression of a marker is the amount of the biomarker, in non-tumor cells in a similar environment or response situation from the same patient that the tumor is derived from. The normal amount of DNA copy number is 2 or diploid.

“Over-expression” and “under-expression” of a biomarker refer to expression of the biomarker of a patient at a greater or lesser level, respectively, than normal level of expression of the biomarker (e.g. more than three-halves-fold, at least two-fold, at least three-fold, greater or lesser level etc.) in a test sample that is greater than the standard error of the assay employed to assess expression. A “significant” expression level may refer to level which either meets or is above or below a pre-determined score for a biomarker set as determined by methods provided herein.

In one embodiment, the general form of a prediction rule consists in the specification of a function of one biomarker potentially including clinical covariates to predict response or non-response, or more generally, predict benefit or lack of benefit in terms of suitably defined clinical endpoints.

The simplest form of a prediction rule consists of an univariate model without covariates, where the prediction is determined by means of a cutoff or threshold. Such a model is utilized in one embodiment in the present invention. This can be phrased in terms of the Heaviside function for a specific cutoff c and a biomarker measurement x, where the binary prediction A or B is to be made, then

If H(x−c)=0 then predict A.

If H(x−c)=1 then predict B.

This is the simplest way of using univariate biomarker measurements in prediction rules. If such a simple rule is sufficient, it allows for a simple identification of the direction of the effect, i.e. whether high or low expression levels are beneficial for the patient.

The situation can be more complicated if clinical covariates need to be considered and/or if multiple biomarkers are used in multivariate prediction rules. For example, for a biomarker X it may be determined in a clinical trial population that high expression levels are associated with a better prognosis (univariate analysis). A closer analysis shows that there are two tumor types in the population, one of which possess a worse prognosis than the other one and at the same time the biomarker expression for this tumor group is generally lower.

As used herein, the terms “protein” and “polypeptide” and “proteinaceous agent” are used interchangeably to refer to a chain of amino acids linked together by peptide bonds which optionally can comprise natural or non-natural amino acids. Optionally, the protein or peptide can comprise other molecules in addition to amino acids. Said chain can be of any length. Polypeptides of the present invention include enzymes related to folate pathways in cells including the selected polypeptides, including wherein the polypeptide includes a growth factor receptor polypeptide, which includes EGFR. The accession numbers of EGFR are as follows: RefSeq (mRNA) NM_{—}005228; RefSeq (protein), NP_{—}005219. Location (UCSC) Chr 7: 55.05-55.24 Mb.

As used herein, nucleotide sequences of the gene products of the above identified selected polypeptides include, but are not limited to, the cDNA, genome-derived DNA and synthetic or semi-synthetic DNA or RNA. The full length gene nucleotide sequence of EGFR is contained in SEQ. ID. NO: 1. [NEED TO GENERATE SEQUENCE]

The term “polynucleotide” is used to mean a polymeric form of nucleotides of any length, which contain deoxyribonucleotides, ribonucleotides, and/or their analogs. The terms “polynucleotide” and “nucleotide” as used herein are used interchangeably. Polynucleotides can have any three-dimensional structure, and can perform any function, known or unknown. The term “polynucleotide” includes double-stranded, single-stranded, and triple-helical molecules. Unless otherwise specified or required, any embodiment of the invention described herein that is a polynucleotide encompasses both the double-stranded form and each of two complementary single-stranded forms known or predicted to make up the double stranded form.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide can be comprised of modified nucleotides, such as methylated nucleotides and nucleotide analogs. Analogs of purines and pyrimidines are known in the art, and include, but are not limited to, aziridinylcytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudouracil, 5-pentynyluracil and 2,6-diaminopurine. The use of uracil as a substitute for thymine in a deoxyribonucleic acid is also considered an analogous form of pyrimidine.

A “fragment” (also called a “region”) of a polynucleotide (i.e., a polynucleotide encoding a SNP) is a polynucleotide comprised of at least 9 contiguous nucleotides of the novel genes. Preferred fragments are comprised of a region encoding at least 5 contiguous amino acid residues, more preferably, at least 10 contiguous amino acid residues, and even more preferably at least 15 contiguous amino acid residues.

The term “recombinant” polynucleotide as used herein intends a polynucleotide of genomic, cDNA, semisynthetic, or synthetic in origin which, by virtue of its origin or manipulation: is not associated with all or a portion of a polynucleotide with which it is associated in nature; is linked to a polynucleotide other than that to which it is linked in nature; or does not occur in nature.

As used herein, reference to a selected gene product, protein or polypeptide in the present invention, including EGFR (SEQ ID NO:1), includes full-length proteins, fusion proteins, or any fragment or homologue of such a protein. The amino acid sequence for EGFR from human are described herein as exemplary growth factor-associated polypeptides and proteins. In addition, and by way of example, a “human EGFR protein” refers to a EGFR, protein (generally including a homologue of a naturally occurring EGFR protein) from a human (Homo sapiens) or to a EGFR protein that has been otherwise produced from the knowledge of the structure (e.g., sequence) and perhaps the function of a naturally occurring EGFR protein from Homo sapiens. In other words, a human EGFR protein includes any EGFR protein that has substantially similar structure and function of a naturally occurring EGFR protein from Homo sapiens or that is a biologically active (i.e., has biological activity) homologue of a naturally occurring EGFR protein from Homo sapiens as described in detail herein. As such, a human EGFR protein can include purified, partially purified, recombinant, mutated/modified and synthetic proteins. According to the present invention, the terms “modification” and “mutation” can be used interchangeably, particularly with regard to the modifications/mutations to the amino acid sequence of EGFR (or nucleic acid sequences) described herein.

As used herein, the term “homologue” is used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue can have enhanced, decreased, or substantially similar properties as compared to the naturally occurring protein or peptide. A homologue can include an agonist of a protein or an antagonist of a protein.

Homologues can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

Homologues can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

According to the present invention, an isolated EGFR protein, including a biologically active homologue or fragment thereof, has at least one characteristic of biological activity of activity a wild-type, or naturally occurring EGFR protein (which can vary depending on whether the homologue or fragment is an agonist, antagonist, or mimic of EGFR, and the isoform EGFR).

Homologues of EGFR, including peptide and non-peptide agonists and antagonists of EGFR (analogues), can be products of drug design or selection and can be produced using various methods known in the art. Such homologues can be referred to as mimetics.

In one embodiment, an EGFR homologue comprises, consists essentially of, or consists of, an amino acid sequence that is at least about 45%, or at least about 50%, or at least about 55%, or at least about 60%, or at least about 65%, or at least about 70%, or at least about 75%, or at least about 80%, or at least about 85%, or at least about 90%, or at least about 95% identical, or at least about 95% identical, or at least about 96% identical, or at least about 97% identical, or at least about 98% identical, or at least about 99% identical (or any percent identity between 45% and 99%, in whole integer increments), to a naturally occurring EGFR amino acid sequence. A homologue of EGFR differs from a reference (e.g., wild-type) EGFR and therefore is less than 100% identical to the reference EGFR at the amino acid level.

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schaaffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) and/or PSI-BLAST with the standard default parameters (Position-Specific Iterated BLAST. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

The term, “primer”, as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the oligonucleotide primer typically contains 15-25 or more nucleotides, although it may contain fewer nucleotides. The factors involved in determining the appropriate length of primer are readily known to one of ordinary skill in the art. In general, the design and selection of primers embodied by the instant invention is according to methods that are standard and well known in the art, see Dieffenbach, C. W., Lowe, T. M. J., Dveksler, G. S. (1995) General Concepts for PCR Primer Design. In: PCR Primer, A Laboratory Manual (Eds. Dieffenbach, C. W, and Dveksler, G. S.) Cold Spring Harbor Laboratory Press, New York, 133-155; Innis, M. A., and Gelfand, D. H. (1990) Optimization of PCRs. In: PCR protocols, A Guide to Methods and Applications (Eds. Innis, M. A., Gelfand, D. H., Sninsky, J. J., and White, T. J.) Academic Press, San Diego, 3-12; Sharrocks, A. D. (1994) The design of primers for PCR. In: PCR Technology, Current Innovations (Eds. Griffin, H. G., and Griffin, A. M, Ed.) CRC Press, London, 5-11.

As used herein, the terms “RNA portion” and “a portion thereof” in context of RNA products of a biomarker of the invention refer to an RNA transcript comprising a nucleic acid sequence of at least 6, at least 9, at least 15, at least 18, at least 21, at least 24, at least 30, at least 60, at least 90, at least 99, or at least 108, or more nucleotides of a RNA product of a biomarker of the invention.

Obtaining a sample of the patient's cancer tissue may be done by any methods known in the art. Bone marrow or lymph node biopsies and analysis of peripheral blood samples for cytogenetic and/or immunologic analysis is standard practice. Frozen tissue specimens may be obtained as well. As used herein a “sample” can be from any organism and can further include, but is not limited to, peripheral blood, plasma, urine, saliva, gastric secretion, feces, bone marrow specimens, primary tumors, metastatic tissue, embedded tissue sections, frozen tissue sections, cell preparations, cytological preparations, exfoliate samples (e.g., sputum), fine needle aspirations, amino cells, fresh tissue, dry tissue, and cultured cells or tissue. It is further contemplated that the biological sample of this invention can also be whole cells or cell organelles (e.g., nuclei). The sample can be unfixed or fixed according to standard protocols widely available in the art.

In some embodiments of the present invention, peripheral blood is drawn, or alternatively, if desired, leukocytes may be isolated by differential gradient separation, using, for example, ficoll-hypaque or sucrose gradient solutions for cell separations, followed by ammonium chloride or hypotonic lysis of remaining contaminating erythrocytes (“Cell Biology: A Laboratory Handbook”, Volumes I-III Cellis, J. E., ed. (1994); “Current Protocols in Immunology” Volumes I-III Coligan J. E., ed. (1994); Stites et al. (eds)). Bone marrow and lymph node biopsies may be processed by collagenase/dispase treatment of the biopsy material, or by homogenization in order to obtain single cell suspensions (“Cell Biology: A Laboratory Handbook”, Volumes I-III Cellis, J. E., ed. (1994); “Current Protocols in Immunology” Volumes I-III Coligan J. E., ed. (1994); Stites et al. (eds)).

The sample can be from a subject or a patient. As utilized herein, the “subject” or “patient” of the methods described herein can be any animal. In a preferred embodiment, the animal of the present invention is a human. In addition, determination of expression patterns is also contemplated for non-human animals which can include, but are not limited to, cats, dogs, birds, horses, cows, goats, sheep, guinea pigs, hamsters, gerbils, mice and rabbits.

The term “cancer,” when used herein refers to or describes the pathological condition, preferably in a mammalian subject, that is typically characterized by unregulated cell growth. Non-limiting cancer types include carcinoma (e.g., adenocarcinoma), sarcoma, myeloma, leukemia, and lymphoma, and mixed types of cancers, such as adenosquamous carcinoma, mixed mesodermal tumor, carcinosarcoma, and teratocarcinoma.

In one embodiment, cancers include solid tumors, in particular, non-small cell lung cancer, head and neck cancer, prostate cancer, and breast cancer. Other cancers include but are not limited to, bladder cancer, lung cancer, colon cancer, rectal cancer, endometrial cancer, ovarian cancer; and melanoma. Specifically included are AIDS-related cancers (e.g., Kaposi's Sarcoma, AIDS-related lymphoma), bone cancers (e.g., osteosarcoma, malignant fibrous histiocytoma of bone, Ewing's Sarcoma, and related cancers), and hematologic/blood cancers (e.g., adult acute lymphoblastic leukemia, childhood acute lymphoblastic leukemia, adult acute myeloid leukemia, childhood acute myeloid leukemia, chronic lymphocytic leukemia, chronic myelogenous leukemia, hairy cell leukemia, adult Hodgkin's disease, childhood Hodgkin's disease, Hodgkin's disease during pregnancy, adult non-Hodgkin's lymphoma, childhood non-Hodgkin's lymphoma, non-Hodgkin's lymphoma during pregnancy, primary central nervous system lymphoma, Waldenstrom's macroglobulinemia, multiple myeloma/plasma cell neoplasm, myelodysplastic syndrome, and myeloproliferative disorders), as well as lymphoblastic lymphomas in which the malignancy occurs in primitive lymphoid progenitors from the thymus; mature or peripheral T-cell neoplasms, including T-cell prolymphocytic leukemia, T-cell granular lymphocytic leukemia, aggressive NK-cell leukemia, cutaneous T-cell lymphoma (Mycosis fungoides/Sezary syndrome), anaplastic large cell lymphoma, T-cell type, enteropathy-type T-cell lymphoma, Adult T-cell leukemia/lymphoma including those associated with HTLV-1, and angioimmunoblastic T-cell lymphoma, and subcutaneous panniculitic T-cell lymphoma; and peripheral T-cell lymphomas that initially involve a lymph node paracortex and never grow into a true follicular pattern.

Also included are brain cancers (e.g., adult brain tumor, childhood brain stem glioma, childhood cerebellar astrocytoma, childhood cerebral astrocytoma, childhood ependymoma, childhood medulloblastoma, supratentorial primitive neuroectodermal and pineal, and childhood visual pathway and hypothalamic glioma), digestive/gastrointestinal cancers (e.g., anal cancer, extrahepatic bile duct cancer, gastrointestinal carcinoid tumor, colon cancer, esophageal cancer, gallbladder cancer, adult primary liver cancer, childhood liver cancer, pancreatic cancer, rectal cancer, small intestine cancer, and gastric cancer), musculoskeletal cancers (e.g., childhood rhabdomyosarcoma, adult soft tissue sarcoma, childhood soft tissue sarcoma, and uterine sarcoma), and endocrine cancers (e.g., adrenocortical carcinoma, gastrointestinal carcinoid tumor, islet cell carcinoma (endocrine pancreas), parathyroid cancer, pheochromocytoma, pituitary tumor, and thyroid cancer).

Also included are neurologic cancers (e.g., neuroblastoma, pituitary tumor, and primary central nervous system lymphoma), eye cancers (e.g., intraocular melanoma and retinoblastoma), genitourinary cancers (e.g., bladder cancer, kidney (renal cell) cancer, penile cancer, transitional cell renal pelvis and ureter cancer, testicular cancer, urethral cancer, Wilms' tumor and other childhood kidney tumors), respiratory/thoracic cancers (e.g., non-small cell lung cancer, small cell lung cancer, malignant mesothelioma, and malignant thymoma), germ cell cancers (e.g., childhood extracranial germ cell tumor and extragonadal germ cell tumor), skin cancers (e.g., melanoma, and merkel cell carcinoma), gynecologic cancers (e.g., cervical cancer, endometrial cancer, gestational trophoblastic tumor, ovarian epithelial cancer, ovarian germ cell tumor, ovarian low malignant potential tumor, uterine sarcoma, vaginal cancer, and vulvar cancer), and unknown primary cancers.

In one embodiment, the sample and the reference cancer are both the same cancer sub-type, i.e., the sample cancer is derived from the same type of cell as the reference cancer. In another embodiment, the reference cancer is any one of or a combination of a cancer or cancerous cell line derived from a solid tumor, in particular, non-small cell lung cancer, head and neck cancer, prostate cancer, and breast cancer, T-cell lymphoma or a multiple myeloma, such as, for example, lymphoblastic lymphomas in which the malignancy occurs in primitive lymphoid progenitors from the thymus; mature or peripheral T-cell neoplasms, including T-cell prolymphocytic leukemia, T-cell granular lymphocytic leukemia, aggressive NK-cell leukemia, cutaneous T-cell lymphoma (Mycosis fungoides/Sezary syndrome), anaplastic large cell lymphoma, T-cell type, enteropathy-type T-cell lymphoma, Adult T-cell leukemia/lymphoma including those associated with HTLV-1, and angioimmunoblastic T-cell lymphoma, and subcutaneous panniculitic T-cell lymphoma; and peripheral T-cell lymphomas that initially involve a lymph node paracortex. As used in the specification and claims of this application, the term “lymphomas” refers to Non-Hodgkins Lymphoma (NHL); diffuse large B-cell lymphoma (DLBCL); follicular lymphoma (FL); Hodgkin's Disease; Burkitt's Lymphoma; cutaneous T-cell lymphoma; primary central nervous system lymphoma, and lymphomatous metastases. In one embodiment of the present invention, this application relates to the use of 10-propargyl-10-deazaminopterin in the treatment of T-cell lymphoma.

In another embodiment of the present invention, the reference cancer or cancerous cell line is a reference cancer or cancerous cell line which is known to have a greater sensitivity to 10-propargyl-10-deazaminopterin. The term, “greater sensitivity,” includes those cancers that are known or are found to have an enhanced response to 10-propargyl-10-deazaminopterin as compared to methotrexate (“MTX.”) Increased sensitivity may be determined by those of skill in the art and may include assessment of effects seen in cell lines derived from that cancer and/or type of cancer, in animal models, such as mouse subcutaneous transplantation models, and therapeutic indicators such as remission or other indicia of reduced tumor burden in patients, such as increased apoptosis, decreased tumor volume, growth inhibition, and other indicia known to those in the art. An enhanced response can include differential effects seen at equivalent doses of, serum concentrations of, or other indicia of equivalence between, MTX and 10-propargyl-10-deazaminopterin.

The selected polypeptides may be quantitated and/or relative amounts determined by any method known in the art for quantitating and/or determining relative amounts of expression levels. The term, “quantitate” or “quantitation” also includes determination of relative amounts of a polypeptide or its transcript. Quantitating transcript RNA or portions thereof of a selected polypeptide is one such method. RNA may be extracted from biological samples via a number of standard techniques (see Current Protocols in Molecular Biology” Volumes I-III Ausubel, R. M., ed. (1994); Ausubel et al., “Current Protocols in Molecular Biology”, John Wiley and Sons, Baltimore, Md. (1989)). Guanidium-based methods for cell lysis enabling RNA isolation, with subsequent cesium chloride step gradients for separation of the RNA from other cellular macromolecules, followed by RNA precipitation and resuspension, is an older, less commonly employed method of RNA isolation (Glisin, Ve. et al (1973) Biochemistry 13: 2633). Alternatively, RNA may be isolated in a single step procedure (U.S. Pat. No. 4,843,155, and Puissant, C. and Houdebine L. M. (1990) Biotechniques 8: 148-149). Single step procedures include the use of Guanidium isothiocyanate for RNA extraction, and subsequent phenol/chloroform/isoamyl alcohol extractions facilitating the separation of total RNA from other cellular proteins and DNA. Commercially available single-step formulations based on the above-cited principles may be employed, including, for example, the use of the TRIZOL reagent (Life Technologies, Gaithersburg, Md.).

According to further features of preferred embodiments of the present invention, monitoring selected polypeptide RNA/gene expression is via a number of standard techniques well described in the art, any of which can be employed to evaluate selected polypeptide expression. These assays comprise Northern blot and dot blot analysis, primer extension, RNase protection, RT-PCR, in-situ hybridization and chip hybridization. Specific selected polypeptide RNA sequences can be readily detected by hybridization of labeled probes to blotted RNA preparations extracted as above. In Northern blot analysis, fractionated RNA is subjected to denaturing agarose gel electrophoresis, which prevents RNA from assuming secondary structures that might inhibit size based separation. RNA is then transferred by capillary transfer to a nylon or nitrocellulose membrane support and may be probed with a labeled oligonucleotide probe complementary to the selected polypeptide sequence (Alwine, et al. (1977). Proc. Natl. Acad. Sci. USA 74: 5350-5354 and Current Protocols in Molecular Biology” Volumes I-III Ausubel, R. M., ed. (1994); Ausubel et al., “Current Protocols in Molecular Biology”, John Wiley and Sons, Baltimore, Md. (1989)).

Alternatively, unfractionated RNA may be immobilized on a nylon or nitrocellulose membrane, and similarly probed for selected polypeptide-specific expression, by Slot/Dot blot analysis. RNA slot/dot blots can be prepared by hand, or alternatively constructed using a manifold apparatus, which facilitates comparing hybridization signals by densitometry scanning (Chomczynski P. (1992) Anal. Biochem. 201: 134-139). Primer extension is an additional means whereby quantification of the RNA may be accomplished. Primer extension provides an additional benefit in mapping the 5′ terminus of a particular RNA, by extending a primer using the enzyme reverse transcriptase. In this case, the primer is an oligonucleotide (or restriction fragment) complementary to a portion of the selected polypeptide mRNA. The primer is end-labeled, and is allowed to hybridize to template selected polypeptide mRNA. Once hybridized, the primer is extended by addition of reverse transcriptase, and incorporation of unlabeled dexoynucleotides to for a single-stranded DNA complementary to template selected polypeptide mRNA. DNA is then analyzed on a sequencing gel, with the length of extended primer serving to map the 5′ position of the mRNA, and the yield of extended product reflecting the abundance of RNA in the sample (Jones et al (1985) Cell 42: 559-572 and Mierendorf R. C. And Pfeffer, D. (1987). Methods Enzymol. 152: 563-566).

RNase protection assays provide a highly sensitive means of quantifying selected polypeptide RNA, even in low abundance. In protection assays, sequence-specific hybridization of ribonucleotide probes complementary to selected polypeptide RNA, with high specific activity are generated, and hybridized to sample RNA. Hybridization reactions are then treated with ribonuclease to remove free probe, leaving intact fragments of annealed probe hybridized to homologous selected polypeptide sequences in sample RNA. Fragments are then analyzed by electrophoresis on a sequencing gel, when appropriately-sized probe fragments are visualized (Zinn K. et al (1983) Cell 34: 865-879 and Melton S. A., et al (1984). Nucl. Acids Res. 12: 7035-7056).

RT-PCR is another means by which selected polypeptide expression is verified. RT-PCR is a particularly useful method for detecting rare transcripts, or transcripts in low abundance. RT-PCR employs the use of the enzyme reverse transcriptase to prepare cDNA from RNA samples, using deoxynucleotide primers complementary to the selected polypeptide mRNA. Once the cDNA is generated, it is amplified through the polymerase chain reaction, by the addition of dexoynucleotides and a DNA polymerase that functions at high temperatures. Through repetitive cycles of primer annealing, incorporation of dexoynucleotides facilitating cDNA extension, followed by strand denaturation, amplification of the desired sequence occurs, yielding an appropriately sized fragment that may be detected by agarose gel electrophoresis. Alternatively, the RT-PCR reaction can be quantified in real-time using techniques known to those skilled in the art. Optimal reverse transcription, hybridization, and amplification conditions will vary depending upon the sequence composition and length(s) of the primers and target(s) employed, and the experimental method selected by the practitioner. Various guidelines may be used to select appropriate primer sequences and hybridization conditions (see, e.g., Sambrook et al., 1989, Molecular Cloning, A Laboratory Manual, (Volumes 1-3) Cold Spring Harbor Press, N.Y.; and Ausubel et al., 1989, Current Protocols in Molecular Biology, Green Publishing Associates and Wiley Interscience, N.Y.).

In-situ hybridization provides another tool for the detection and localization of cell/tissue specific selected polypeptide RNA expression. Labeled anti-sense RNA probes are hybridized to mRNAs in cells singly, or in processed tissue slices, which are immobilized on microscope glass slides (In Situ Hybridization: Medical Applications (eds. G. R. Coulton and J. de Belleroche), Kluwer Academic Publishers, Boston (1992); In Situ Hybridization: In Neurobiology; Advances in Methodology (eds. J. H. Eberwine, K. L. Valentino, and J. D. Barchas), Oxford University Press Inc., England (1994); and In Situ Hybridization: A Practical Approach (ed. D. G. Wilkinson), Oxford University Press Inc., England (1992)). Numerous non-isotopic systems have been developed to visualize labeled DNA probes including; a) fluorescence-based direct detection methods, b) the use of digoxigenin- and biotin-labeled DNA probes coupled with fluorescence detection methods, and c) the use of digoxigenin- and biotin-labeled DNA probes coupled with antibody-enzyme detection methods. When fluorescence-labeled anti-sense RNA probes are hybridized to cellular RNA, the hybridized probes can be viewed directly using a fluorescence microscope. Direct fluorochrome-labeling of the nucleic acid probes eliminate the need for multi-layer detection procedures (e.g., antibody-based-systems), which allows fast processing and also reduces non-specific background signals, hence providing a versatile and highly sensitive means of identifying selected polypeptide gene expression.

Chip hybridization utilizes selected polypeptide-specific oligonucleotides attached to a solid substrate, which may consist of a particulate solid phase such as nylon filters, glass slides or silicon chips [Schena et al. (1995) Science 270:467-470] designed as a microarray. Microarrays are known in the art and consist of a surface to which probes that correspond in sequence to gene products (such as cDNAs) can be specifically hybridized or bound at a known position for the detection of selected polypeptide gene expression. Quantification of the hybridization complexes is well known in the art and may be achieved by any one of several approaches. These approaches are generally based on the detection of a label or marker, such as any radioactive, fluorescent, biological or enzymatic tags or labels of standard use in the art. A label can be applied to either the oligonucleotide probes or the RNA derived from the biological sample.

In general, mRNA quantification is preferably effected alongside a calibration curve so as to enable accurate mRNA determination. Furthermore, quantifying transcript(s) originating from a biological sample is preferably effected by comparison to a normal sample, which sample is characterized by normal expression pattern of the examined transcript(s).

Selected polypeptide expression may also be evaluated at the level of protein expression, either by demonstration of the presence of the protein, or by its activity, with activity herein referring to the enzymatic activity of the selected polypeptide enzyme. Methods for monitoring specific polypeptide protein expression include but are not limited to the following methods discussed below. Anti-selected polypeptide-antibodies for use in selected polypeptide-specific protein detection are readily generated by methods known in the art and include both polyclonal and monoclonal antibodies. The antibodies preferably bind to both native and denatured selected polypeptides and may be detected by several well-known assays in the art, including ELISA, RIA, light emission immunoassays, Western blot analysis, immunofluorescence assays, immunohistochemistry and FACS analysis.

Enzyme linked immunosorbant (ELISA) assays and radioimmunoassays (RIA) follow similar principles for detection of specific antigens, in this case, selected polypeptides. In RIA a selected polypeptide-specific antibody is radioactively labeled, typically with ¹²⁵I. In ELISA assays a selected polypeptide-specific antibody is chemically linked to an enzyme. Selected polypeptide-specific capturing antibody is immobilized onto a solid support. Unlabelled specimens, e.g., protein extracts from biopsy or blood samples are then incubated with the immobilized antibody under conditions where non-specific binding is blocked, and unbound antibody and/or protein removed by washing. Bound selected polypeptide is detected by a second selected polypeptide-specific labeled antibody. Antibody binding is measured directly in RIA by measuring radioactivity, while in ELISA binding is detected by a reaction converting a colorless substrate into a colored reaction product, as a function of linked-enzyme activity. Changes can thus readily be detected by spectrophotometry (Janeway C. A. et al (1997). “Immunology” 3rd Edition, Current Biology Ltd., Garland Publishing Inc.; “Cell Biology: A Laboratory Handbook”, Volumes I-III Cellis, J. E., ed. (1994); “Current Protocols in Immunology” Volumes I-III Coligan J. E., ed. (1994); Stites et al. (eds)). Both assays therefore provide a means of quantification of selected polypeptide protein content in a biological sample.

Selected polypeptide protein expression may also be detected via light emission immunoassays. Much like ELISA and RIA, in light emission immunoassays the biological sample/protein extract to be tested is immobilized on a solid support, and probed with a specific label, labeled anti-selected polypeptide antibody. The label, in turn, is luminescent, and emits light upon binding, as an indication of specific recognition. Luminescent labels include substances that emit light upon activation by electromagnetic radiation, electro chemical excitation, or chemical activation and may include fluorescent and phosphorescent substances, scintillators, and chemiluminescent substances. The label can be a part of a catalytic reaction system such as enzymes, enzyme fragments, enzyme substrates, enzyme inhibitors, coenzymes, or catalysts; part of a chromogen system such as fluorophores, dyes, chemiluminescers, luminescers, or sensitizers; a dispersible particle that can be non-magnetic or magnetic, a solid support, a liposome, a ligand, a receptor, a hapten radioactive isotope, and so forth (U.S. Pat. No. 6,410,696, U.S. Pat. No. 4,652,533 and European Patent Application No. 0,345,776), and provide an additional, highly sensitive method for detection of selected polypeptide protein expression.

Western blot analysis is another means of assessing selected polypeptide content in a biological sample. Protein extracts from biological samples of, for example, hematopoietic cells, are solubilized in a denaturing ionizing environment, and aliquots are applied to polyacrylamide gel matrixes. Proteins separate based on molecular size properties as they migrate toward the anode. Antigens are then transferred to nitrocellulose, PVDF or nylon membranes, followed by membrane blocking to minimize non-specific binding. Membranes are probed with antibodies directly coupled to a detectable moiety, or are subsequently probed with a secondary antibody containing the detectable moiety. Typically the enzymes horseradish peroxidase or alkaline phosphatase are coupled to the antibodies, and chromogenic or luminescent substrates are used to visualize activity (Harlow E. et al (1998) Immunoblotting. In Antibodies: A Laboratory Manual, pp. 471-510 CSH Laboratory, cold Spring Harbor, N.Y. and Bronstein I. Et al. (1992) Biotechniques 12: 748-753). Unlike RIA, ELISA, light emission immunoassays and immunoblotting, which quantify selected polypeptide content in whole samples, immunofluorescence/immunocytochemistry may be used to detect proteins in a cell-specific manner, though quantification is compromised.

In another embodiment, the present invention includes a method to modulate the expression of a growth factor polypeptide in a patient's cancer comprising administering to a patient an effective amount of 10-propargyl-10-deazaminopterin. In one embodiment, the patient's cancer is a solid tumor, in particular, non-small cell lung cancer, head and neck cancer, prostate cancer, and breast cancer, or a lymphoma; in one embodiment, the patient's cancer is a T-cell lymphoma; in another embodiment, the patient's cancer is NSCLC. The modulation can occur in vitro and/or in vivo. Modulation includes both up-regulation and down-regulation. As used herein, the term “up regulated” or “increased level of expression” in the context of this invention refers to a sequence corresponding to a gene which is expressed wherein the measure of the quantity of the sequence demonstrates an increased level of expression of the gene in the patient as compared to prior to administration of 10-propargyl-10-deazaminopterin, and can be observed at any point in treatment with 10-propargyl-10-deazaminopterin. An “increased level of expression” according to the present invention, is an increase in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more. “Down regulation” or “decreased level of expression” in the context of this invention refers to a sequence corresponding to a gene which is expressed wherein the measure of the quantity of the sequence demonstrates a decreased level of expression of the gene in the patient as compared to prior to administration of 10-propargyl-10-deazaminopterin, and can be observed at any point in treatment with 10-propargyl-10-deazaminopterin. A “decreased level of expression” according to the present invention, is a decrease in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more. In one embodiment, the 10-propargyl-10-deazaminopterin is substantially free of 10-deazaminopterin.

In one embodiment, kits are provided for measuring a RNA product of a growth factor receptor polypeptide which comprise materials and reagents that are necessary for measuring the expression of the RNA product. For example, a microarray or RT-PCR kit may be used and contain only those reagents and materials necessary for measuring the levels of RNA products of any number of up to at least 1, at least 2, at least 3, at least 4, at least 5, at least 6, all or any combination of the growth factor polypeptides. Alternatively, in some embodiments, the kits can comprise materials and reagents that are not limited to those required to measure the levels of RNA products of any number of up to 1, 2, 3, 4, 5, 6, all or any combination of the growth factor receptor polypeptides. For example, a microarray kit may contain reagents and materials necessary for measuring the levels of RNA products any number of up to 1, 2, 3, 4, 5, 6, all or any combination of the growth factor receptor polypeptide s, in addition to reagents and materials necessary for measuring the levels of the RNA products of any number of up to at least 1, at least 2, at least 3, at least 4, at least 5, at least 6, or more genes other than the growth factor receptor polypeptides. In a specific embodiment, a microarray or RT-PCR kit contains reagents and materials necessary for measuring the levels of RNA products of any number of up to at least 1, at least 2, at least 3, at least 4, at least 5, at least 6, all or any combination of the growth factor receptor polypeptide s, and any number of up to 1, 2, 3, 4, 5, 10 or more genes that are not growth factor receptor polypeptides.

For nucleic acid microarray kits, the kits generally comprise probes attached to a support surface. The probes may be labeled with a detectable label. In a specific embodiment, the probes are specific for the 5′ region, the 3′ region, the internal coding region, an exon(s), an intron(s), an exon junction(s), or an exon-intron junction(s), of any number of up to 1, 2, 3, 4, 5, 6, all or any combination of the growth factor receptor polypeptide s. The microarray kits may comprise instructions for performing the assay and methods for interpreting and analyzing the data resulting from the performance of the assay. The kits may also comprise hybridization reagents and/or reagents necessary for detecting a signal produced when a probe hybridizes to a target nucleic acid sequence. Generally, the materials and reagents for the microarray kits are in one or more containers. Each component of the kit is generally in its own a suitable container.

For RT-PCR kits, the kits generally comprise pre-selected primers specific for particular RNA products (e.g., an exon(s), an intron(s), an exon junction(s), and an exon-intron junction(s)) of any number of up to 1, 2, 3, 4, 5, 6, all or any combination of growth factor receptor polypeptide s. The RT-PCR kits may also comprise enzymes suitable for reverse transcribing and/or amplifying nucleic acids (e.g., polymerases such as Taq), and deoxynucleotides and buffers needed for the reaction mixture for reverse transcription and amplification. The RT-PCR kits may also comprise probes specific for any number of up to 1, 2, 3, 4, 5, 6, all or any combination of the growth factor receptor polypeptides. The probes may or may not be labeled with a detectable label (e.g., a fluorescent label). Each component of the RT-PCR kit is generally in its own suitable container. Thus, these kits generally comprise distinct containers suitable for each individual reagent, enzyme, primer and probe. Further, the RT-PCR kits may comprise instructions for performing the assay and methods for interpreting and analyzing the data resulting from the performance of the assay.

For antibody based kits, the kit can comprise, for example: (1) a first antibody (which may or may not be attached to a support) which binds to protein of interest (e.g., a protein product of any number of up to 1, 2, 3, 4, 5, 6, all or any combination of the growth factor receptor polypeptides); and, optionally, (2) a second, different antibody which binds to either the protein, or the first antibody and is conjugated to a detectable label (e.g., a fluorescent label, radioactive isotope or enzyme). The antibody-based kits may also comprise beads for conducting an immunoprecipitation. Each component of the antibody-based kits is generally in its own suitable container. Thus, these kits generally comprise distinct containers suitable for each antibody. Further, the antibody-based kits may comprise instructions for performing the assay and methods for interpreting and analyzing the data resulting from the performance of the assay.

The present invention also relates to methods and compositions effective to treat non-small cell lung cancer and other cancers as described elsewhere herein. 10-propargyl-10-deazaminopterin, in combination with an EGFR inhibitor, including erlotinib, has been found to be effective at decreasing tumor growth in a xenograft mouse model for NSCLC at a well-tolerated dose, in a synergistic manner. Dosing with a combination of 10-propargyl-10-deazaminopterin and erlotinib can optionally be carried out in a sub-therapeutic dosage relative to each individual component, providing greater than additive inhibitory effects upon the growth of the tumor.

Combination therapy using 10-propargyl-10-deazaminopterin and Tarceva® has been tested in model systems for efficacy against NSCLC. Specifically, the effect of 10-propargyl-10-deazaminopterin alone and in combination with Tarceva® was tested in the A549 xenograft mouse model. A549 is a human lung cancer cell line, initiated in 1972 by D. J. Giard et al. through explant culture of lung carcinomatous tissue from a 58 yr-old Caucasian male, available from the American Type Culture Collection (ATCC). Surprisingly, the study found that 10-propargyl-10-deazaminopterin at 2 mg/kg, IP, in combination with 50 mg/kg Tarceva®, PO, is significantly more effective at controlling A549 tumor growth, compared to control and Tarceva® alone treated groups, and also compared with the 10-propargyl-10-deazaminopterin alone groups. 10-propargyl-10-deazaminopterin alone at both doses tested (1 or 2 mg/kg), showed a trend toward controlling tumor growth compared to vehicle alone. Tarceva® is customarily used at a dose of 100 mg/kg.

The results disclosed herein show that combining 50 mg/kg Tarceva® with 2 mg/kg 10-propargyl-10-deazaminopterin significantly reduced the in vivo growth of A549 NSCLC cell xenografts. Of note, the administration of 2 mg/kg 10-propargyl-10-deazaminopterin alone also inhibited the growth of the A549 xenografts in nude mice during the study period which lasted for 35 days, which is in agreement with the in vitro activity of this compound on the growth of A549 cells. In vivo, at a well-tolerated dose and schedule, the 10-propargyl-10-deazaminopterin-Tarceva® combination was more effective in decreasing tumor growth than either agent alone. Collectively, these results show that 10-propargyl-10-deazaminopterin is effective in inhibiting the in vitro and in vivo growth of NSCLC alone and in a combination with Tarceva®, in the A549 lung tumor model.

Accordingly, in one embodiment, the present invention provides a pharmaceutical composition comprising an EGFR kinase inhibitor and 10-propargyl-10-deazaminopterin in a pharmaceutically acceptable carrier. In one embodiment, the EGFR kinase inhibitor is erlotinib, as discussed in more detail hereinbelow. In one embodiment of the invention, the composition comprises “highly purified” 10-propargyl-10-deazaminopterin.

10-propargyl-10-deazaminopterin and other agents such as erlotinib may be concurrently administered or utilized in combination as part of a common treatment regimen, in which the 10-propargyl-10-deazaminopterin and the other agent(s) are administered at different times. For example, the other agent may be administered before, immediately afterward or after a period of time (for example 24 hours) relative to the 10-propargyl-10-deazaminopterin administration. Thus, for purposes of this application, the term administering refers generally to concurrent administration or to sequential administration of the drugs and in either order in a parallel treatment regimen with or without a separation in time between the drugs unless otherwise specified.

As used herein, the term “EGFR kinase inhibitor” also refers to any EGFR kinase inhibitor that is currently known in the art or that will be identified in the future, and includes any chemical entity that, upon administration to a patient, results in inhibition of a biological activity associated with activation of the EGF receptor in the patient, including any of the downstream biological effects otherwise resulting from the binding to EGFR of its natural ligand. Such EGFR kinase inhibitors include any agent that can block EGFR activation or any of the downstream biological effects of EGFR activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the EGFR receptor, thereby making the receptor inaccessible to its natural ligand so that its normal biological activity is prevented or reduced. Alternatively, such an inhibitor can act by modulating the dimerization of EGFR polypeptides, or interaction of EGFR polypeptide with other proteins, or enhance ubiquitination and endocytotic degradation of EGFR. EGFR kinase inhibitors include but are not limited to low molecular weight inhibitors, antibodies or antibody fragments, antisense constructs, small inhibitory RNAs (i.e. RNA interference by dsRNA; RNAi), and ribozymes. In a preferred embodiment, the EGFR kinase inhibitor is a small organic molecule or an antibody that binds specifically to the human EGFR.

More specifically, EGFR kinase inhibitors can include, for example, quinazoline EGFR kinase inhibitors, pyrido-pyrimidine EGFR kinase inhibitors, pyrimido-pyrimidine EGFR kinase inhibitors, pyrrolo-pyrimidine EGFR kinase inhibitors, pyrazolo-pyrimidine EGFR kinase inhibitors, phenylamino-pyrimidine EGFR kinase inhibitors, oxindole EGFR kinase inhibitors, indolocarbazole EGFR kinase inhibitors, phthalazine EGFR kinase inhibitors, isoflavone EGFR kinase inhibitors, quinalone EGFR kinase inhibitors, and tyrphostin EGFR kinase inhibitors, such as those described in the following patent publications, and all pharmaceutically acceptable salts and solvates of said EGFR kinase inhibitors: International Patent Publication Nos. WO 96/33980, WO 96/30347, WO 97/30034, WO 97/30044, WO 97/38994, WO 97/49688, WO 98/02434, WO 97/38983, WO 95/19774, WO 95/19970, WO 97/13771, WO 98/02437, WO 98/02438, WO 97/32881, WO 98/33798, WO 97/32880, WO 97/3288, WO 97/02266, WO 97/27199, WO 98/07726, WO 97/34895, WO 96/31510, WO 98/14449, WO 98/14450, WO 98/14451, WO 95/09847, WO 97/19065, WO 98/17662, WO 99/35146, WO 99/35132, WO 99/07701, and WO 92/20642; European Patent Application Nos. EP 520722, EP 566226, EP 787772, EP 837063, and EP 682027; U.S. Pat. Nos. 5,747,498, 5,789,427, 5,650,415, and 5,656,643; and German Patent Application No. DE 19629652. Additional non-limiting examples of low molecular weight EGFR kinase inhibitors include any of the EGFR kinase inhibitors described in Traxler, P., 1998, Exp. Opin. Ther. Patents 8(12):1599-1625.

Specific preferred examples of low molecular weight EGFR kinase inhibitors that can be used according to the present invention include [6,7-bis(2-methoxyethoxy)-4-quinazolin-4-yl]-(3-ethynylphenyl)amine (also known as OSI-774, erlotinib, or Tarceva® (erlotinib HCl); OSI Pharmaceuticals/Genentech/Roche) (U.S. Pat. No. 5,747,498; International Patent Publication No. WO 01/34574, and Moyer, J. D. et al. (1997) Cancer Res. 57:4838-4848); C₁₋₁₀₃₃ (formerly known as PD183805; Pfizer) (Sherwood et al., 1999, Proc. Am. Assoc. Cancer Res. 40:723); PD-158780 (Pfizer); AG-1478 (University of California); CGP-59326 (Novartis); PKI-166 (Novartis); EKB-569 (Wyeth); GW-2016 (also known as GW-572016 or lapatinib ditosylate; GSK); and gefitinib (also known as ZD1839 or Iressa™; Astrazeneca) (Woodburn et al., 1997, Proc. Am. Assoc. Cancer Res. 38:633). A particularly preferred low molecular weight EGFR kinase inhibitor that can be used according to the present invention is [6,7-bis(2-methoxyethoxy)-4-quinazolin-4-yl]-(3-ethynylphenyl)amine (i.e. erlotinib), its hydrochloride salt (i.e. erlotinib HCl, Tarceva®), or other salt forms (e.g. erlotinib mesylate).

Antibody-based EGFR kinase inhibitors include any anti-EGFR antibody or antibody fragment that can partially or completely block EGFR activation by its natural ligand. Non-limiting examples of antibody-based EGFR kinase inhibitors include those described in Modjtahedi, H., et al., 1993, Br. J. Cancer 67:247-253; Teramoto, T., et al., 1996, Cancer 77:639-645; Goldstein et al., 1995, Clin. Cancer Res. 1:1311-1318; Huang, S. M., et al., 1999, Cancer Res. 15:59(8):1935-40; and Yang, X., et al., 1999, Cancer Res. 59:1236-1243. Thus, the EGFR kinase inhibitor can be monoclonal antibody Mab E7.6.3 (Yang, X. D. et al. (1999) Cancer Res. 59:1236-43), or Mab C225 (ATCC Accession No. HB-8508), or an antibody or antibody fragment having the binding specificity thereof. Suitable monoclonal antibody EGFR kinase inhibitors include, but are not limited to, IMC-C225 (also known as cetuximab or Erbitux™; Imclone Systems), ABX-EGF (Abgenix), EMI 72000 (Merck KgaA, Darmstadt), RH3 (York Medical Bioscience Inc.), and MDX-447 (Medarex/Merck KgaA). The invention also encompasses a pharmaceutical composition that is comprised of an EGFR kinase inhibitor and 10-propargyl-10-deazaminopterin combination in combination with a pharmaceutically acceptable carrier.

The present invention, accordingly, provides a method for the treatment of cancer, including non-small cell lung cancer, in a patient in need thereof, comprising administering to a patient either simultaneously or sequentially a therapeutically effective amount of a combination comprising an EGFR kinase inhibitor and 10-propargyl-10-deazaminopterin. In one embodiment, an EGFR kinase inhibitor and/or the 10-propargyl-10-deazaminopterin is administered in an amount that provides for a synergistic anti-tumor effect. In another embodiment of the present invention, an EGFR kinase inhibitor and/or the 10-propargyl-10-deazaminopterin is administered in an amount that is subtherapeutic with respect to the individual components. In one embodiment, the EGFR kinase inhibitor is erlotinib.

The amount of EGFR kinase inhibitor administered and the timing of EGFR kinase inhibitor administration will depend on the type (species, gender, age, weight, smoker/non-smoker, etc.) and condition of the patient being treated, the severity of the disease or condition being treated, and on the route of administration. For example, small molecule EGFR kinase inhibitors can be administered to a patient in doses ranging from 0.001 to 100 mg/kg of body weight per day or per week in single or divided doses, or by continuous infusion (see for example, International Patent Publication No. WO 01/34574). In particular, erlotinib can be administered to a patient in doses ranging from 5-200 mg per day, or 100-1600 mg per week, in single or divided doses, or by continuous infusion. Another dose is 150 mg/day.

In one embodiment, an EGFR kinase inhibitor, including erlotinib, may be administered in either a therapeutic or subtherapeutic amount for the treatment of cancer, including NSCLC. In one embodiment, an EGFR kinase inhibitor, including erlotinib, is administered in a generally subtherapeutic amount of between about 1 mg/kg and about 95 mg/kg for the duration of the treatment regimen. The treatment regimen, in one embodiment, is 35 days. An EGFR kinase inhibitor, including erlotinib, may also be administered in an amount of between about 25 mg/kg and about 75 mg/kg or about 50 mg/kg. Therapeutic amounts of an EGFR kinase inhibitor, including erlotinib, may also be used, including amounts of about 100 mg/kg or greater.

For purposes of the present invention, “co-administration of” and “co-administering” 10-propargyl-10-deazaminopterin with an EGFR kinase inhibitor (both components referred to hereinafter as the “two active agents”) refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. 10-propargyl-10-deazaminopterin can be administered prior to, at the same time as, or subsequent to administration of the EGFR kinase inhibitor, or in some combination thereof. Where the EGFR kinase inhibitor is administered to the patient at repeated intervals, e.g., during a standard course of treatment, 10-propargyl-10-deazaminopterin can be administered prior to, at the same time as, or subsequent to, each administration of the EGFR kinase inhibitor, or some combination thereof, or at different intervals in relation to the EGFR kinase inhibitor treatment, or in a single dose prior to, at any time during, or subsequent to the course of treatment with the EGFR kinase inhibitor.

Unless otherwise indicated, all numbers expressing quantities of ingredients, dimensions reaction conditions and so forth used in the specification and claims are to be understood as being modified in all instances by the term “about”.

In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

The following examples are provided for illustrative purposes only and are not intended to limit the scope of the invention.

### Example 1

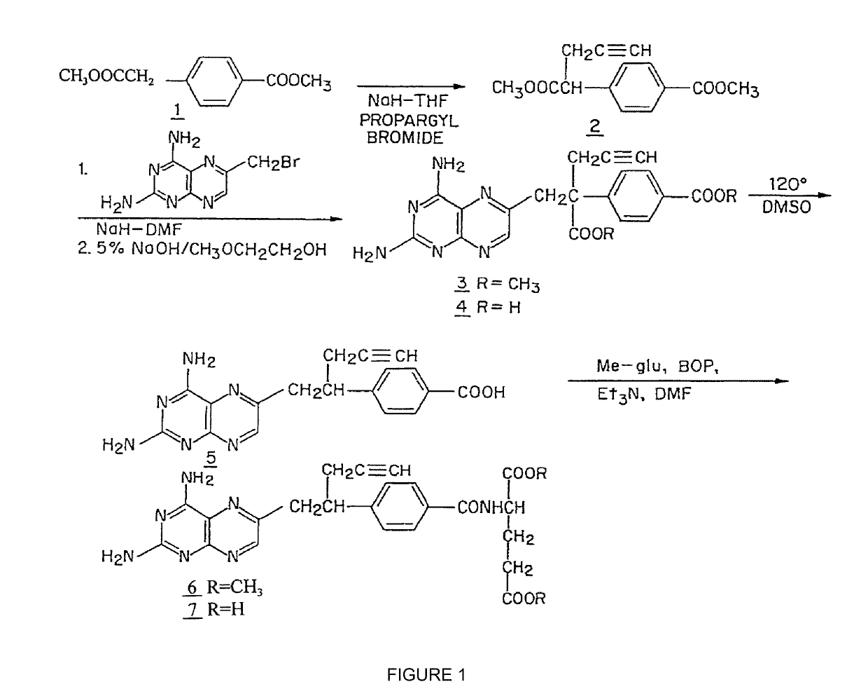

FIG. 1 shows a synthetic scheme useful in preparing 10-propargyl-10-deazaminopterin. A mixture of 60% NaH in oil dispersion (1.06 g, 26.5 mmol) in 18 mL of sieve-dried THF was cooled to 0° C. The cold mixture was treated with a solution of homoterephthalic acid dimethyl ester (5.0 g, 24 mmol. compound 1 in FIG. 1) in dry THF (7 mL), and the mixture was stirred for 1 hour at 0° C. Propargyl bromide (26.4 mmol) was added, and the mixture was stirred at 00° C. for an additional 1 hour, and then at room temperature for 16 hours. The resulting mixture was treated with 2.4 mL of 50% acetic acid and then poured into 240 mL of water. The mixture was extracted with ether (2×150 mL). The ether extracts were combined, dried over Na₂SO₄, and concentrated to an orange-yellow oil. Chromatography on silica gel (600 mL of 230-400 mesh) with elution by cyclohexane-EtOAc (8:1) gave the product α-propargylhomoterephthalic acid dimethyl ester (compound 2) as a white solid (4.66) which appeared by TLC (cyclohexane-EtOAc, 3:1) to be homogeneous. Mass spectral data on this product, however, showed it to be a mixture of the desired product 2, and the dipropargylated compound. No starting material 1 was detected. HPLC shows the ratio of mono- to di-propargylated products to be about 3:1. Since the dipropargylated product, unlike compound 1, cannot produce an unwanted coproduct in the next step of the reaction, this material was suitable for conversion to compound 3. Absence of starting compound 1 in the product used to proceed in the synthesis is very important in order to avoid the sequential formation of 10-dAM during the transformations lading to the final product, because complete removal from 10-dAM from 10-propargyl-10-deazaminopterin is very difficult.

A mixture was formed by combining 0.36 g of a 60% NaH (9 mmol) in oil dispersion with 10 mL of dry DMF and cooled to 0-5° C. The cold mixture was treated drop-wise with a solution of the product of the first reaction (compound 2) (2.94 g, 12 mmol) in 10 mL dry DMF and then stirred at 0° C. for 30 minutes. After cooling to −25° C., a solution of 2,4,diamino-6-(bromomethyl)-pteridine hydrobromide-0.2 2-propanol (1.00 g, 2.9 mmol) in 10 mL dry DMF was added drop-wise while the temperature was maintained near −25° C. The temperature of the stirred mixture was allowed to rise to −10° C. over a period of 2 hours. After an additional 2 hours at −10° C., the temperature was allowed to rise to 20° C., stifling at room temperature was continued for 2 hours longer. The reaction was then adjusted to pH 7 by addition of solid CO₂, After concentration in vacuo to remove solvent, the residue was stirred with diethyl ether and the ether insoluble material was collected, washed with water, and dried in vacuo to give 1.49 g of a crude product. This crude product was dissolved in CHCl₃-MeOH (10:1) for application to a silica gel column. Elution by the same solvent system afforded 10-propargyl-10-carbomethoxy-4-deoxy-4-a-mino-10-deazapteroic acid methyl ester (compound 3) which was homogenous to TLC in 40% yield (485 mg).

A stirred suspension of compound 3 (400 mg, 0.95 mmol) in 2-methoxyethanol (5 mL) was treated with water (5 mL) and then 10% sodium hydroxide solution (3.9 mL). The mixture was stirred as room temperature for 4 hours, during which time solution occurred. The solution was adjusted to pH 8 with acetic acid and concentrated under high vacuum. The resulting residue was dissolved in 15 mL of water and acidified to pH 5.5-5.8 resulting in formation of a precipitate. The precipitate was collected, washed with water and dried in vacuo to recover 340 mg of compound 4 (91% yield). HPLC analysis indicated a product purity of 90%.

Compound 4 (330 mg) was decarboxylated by heating in 15 mL DMSO at 115-120° C. for 10 minutes. A test by HPLC after 10 minutes confirmed that the conversion was essentially complete. DMSO was removed by distillation in vacuo (bath at 40° C.). The residue was stirred with 0.5 N NaOH to give a clear solution, Acidification to pH 5.0 with 1N HCl gave 10-propargyl-4-deoxy-4-amino-10-deazapteroic acid (compound 5) as a yellow solid in 70% yield. HPLC indicated product purity at this stage as 90%.

Compound 5 (225 mg, 0.65 mmol) was coupled with dimethyl L-glutamate hydrochloride (137 mg, 0.65 mmol) using BOP reagent (benzotriazole-1-yloxytris(dimethylamino) phosphonium hexafluorophosphate (287 mg, 0.65 mmol, Aldrich Chemical Co.) in DMF (10 mL) containing triethylamine (148 mg, 1.46 mmol). The mixture was stirred for 3 hours at 20-25° C. and then evaporated to dryness. The residue was stirred with water, and the water-insoluble crude product was collected and dried in vacuo. The crude product (350 mg) was purified by silica gel chromatography with elution by CHCl₃-MeOH (10:1) containing triethylamine (0.25% by volume) to recover 165 mg of 10-propargyl-10-deazaminopterin dimethyl ester (compound 6, 50% yield) which was homogeneous to TLC(CHCl₃-MeOH 5:1).

Compound 6 (165 mg, 0.326 mmol) was suspended in 10 mL stirred MeOH to which 0.72 mL (0.72 meq) 1N NaOH was added. Stirring at room temperature was continued until solution occurred after a few hours. The solution was kept at 20-25°. for 8 hours, then diluted with 10 mL water. Evaporation under reduced pressure removed the methanol, and the concentrated aqueous solution was left at 20-25° C. for another 24 hours. HPLC then showed the ester hydrolysis to be complete. The clear aqueous solution was acidified with acetic acid to pH 4.0 to precipitate 10-propargyl-10-deazaminopterin as a pale yellow solid, The collected, water washed and dried in vacuo product weighed 122 mg (79% yield). Assay by elemental analysis, proton NMR and mass spectroscopy were entirely consistent with the assigned structure. HPLC analysis indicated purity of 98% and established the product to be free of 10-deazaminopterin.

In this case, the amount of 10-propargyl-10-deazaminopterin (as determined by HPLC peak area) approaches 98%, and the peak corresponding to 10-deazaminopterin is not detected by the processing software although there is a minor baseline ripple in this area.

### Example 2

To explore the activity of pralatrexate across different solid tumor types, 15 human solid tumor cell lines were investigated for their sensitivity to the cytotoxic activity of pralatrexate.

Materials and Methods: Cell Lines

A panel of colon (HT29, HCT116, COL0205, HCC2998), breast (MCF7, MDA-MB-435), lung (HOP62, HOP92), ovarian (OVCAR3, IGROV1), prostate (DU145, PC3), and head and neck (SCC61, HEP2, SQ20B) human cancer cell lines was purchased from the ATCC (Rockville, Md.) and National Cancer Institute collections. Cells were grown as monolayers in RPMI medium supplemented with 10% fetal calf serum, 2 mM glutamine, 100 units ml⁻¹ penicillin and 100 μM ml⁻¹ streptomycin.

Cell Cytotoxicity Assays

All the data generated was the result of three separate experiments performed in duplicate. Cell viability was determined using the MTT assay, which was carried out as described previously (Hansen, 1989). Briefly, cells were seeded in 96-well plates at a density of 2×10³ cells well⁻¹. Cells were incubated for 120 hours and then 0.4 mg ml⁻¹ of MTT dye (3-[4,5-dimethylthiazol-2-yl]-2,5-diphenyltetrazolium bromide was added for 4 hours at 37° C. The monolayer was suspended in 0.1 ml of DMSO and the absorbance at 560 nm was measured using a microplate reader. Positive and negative controls included wells with untreated cells or medium containing MTT with no cells, respectively. The conversion of yellow water-soluble tetrazolium MTT into purple insoluble formazan is catalyzed by mitochondrial dehydrogenases and is used to estimate the number of viable cells. The control value corresponding to untreated cells was taken as 100% and the viability of treated samples was expressed as a percentage of the control. IC₅₀ values were determined as concentrations that reduced cell viability by 50%.

For single agent studies, cells were seeded and allowed to settle for 24 hours prior to treatment with increasing concentrations of 10-propargyl-10-deazaminopterin for 72 h. After incubation, the cells were allowed to recover in compound-free medium for 48 h, prior to determination of growth inhibition using the MTT assay.

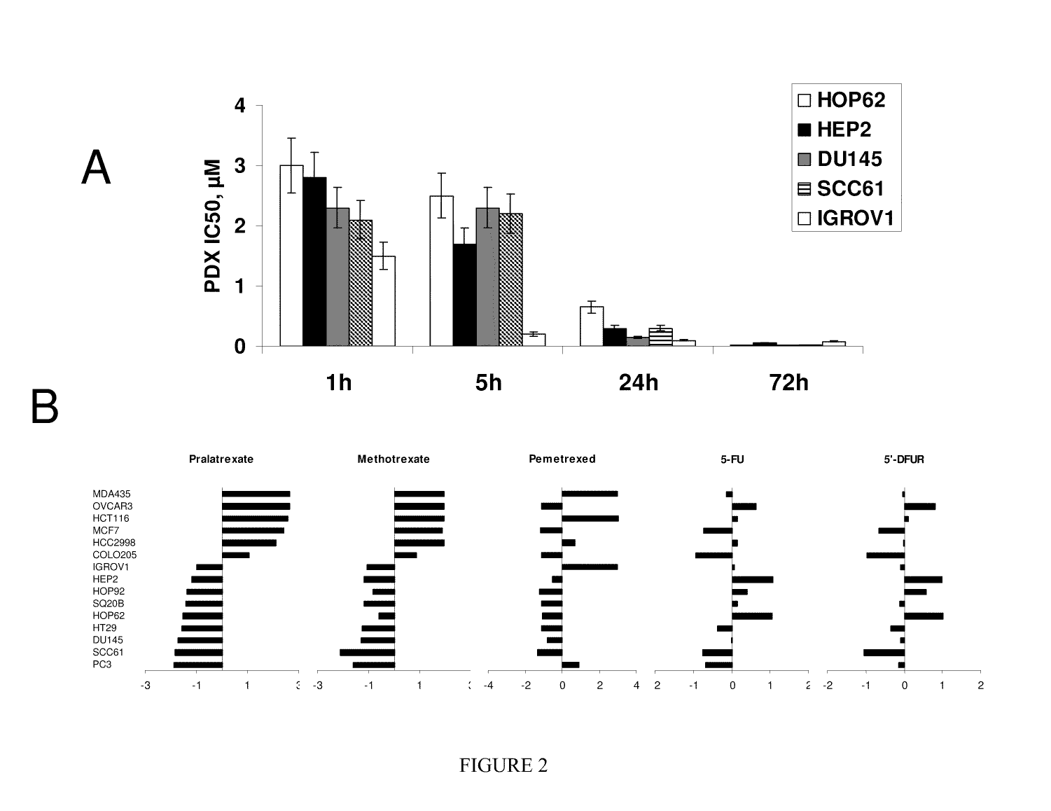

FIG. 2 shows the relative sensitivity to pralatrexate of the 15 human cancer cell lines tested. Nine of the cell lines were found to be sensitive to the cytotoxic activity of pralatrexate (IC₅₀<0.1 μM), whereas 6 of the cell lines were found to be relatively resistant (IC₅₀>9 μM).

### Example 3

In order to establish potential correlations of pralatrexate sensitivity and resistance with expression of genes involved in apoptosis, cell cycle regulation and growth factor pathway signaling, mRNA expression of genes of interest were analyzed using real-time polymerase chain reaction (RT-PCR).

RT-PCR.

The theoretical and practical aspects of quantitative RT-PCR using the ABI Prism 7900 Sequence Detection System (Perkin-Elmer Applied Biosystems, Foster City, Calif., USA) are known to those skilled in the art. Results were expressed as n-fold differences in target gene expression relative to the TBP gene (an endogenous RNA control) and relative to a calibrator (1× sample), consisting of the cell line sample from the tested series that contained the smallest amount of target gene mRNA. Experiments were performed in duplicate.

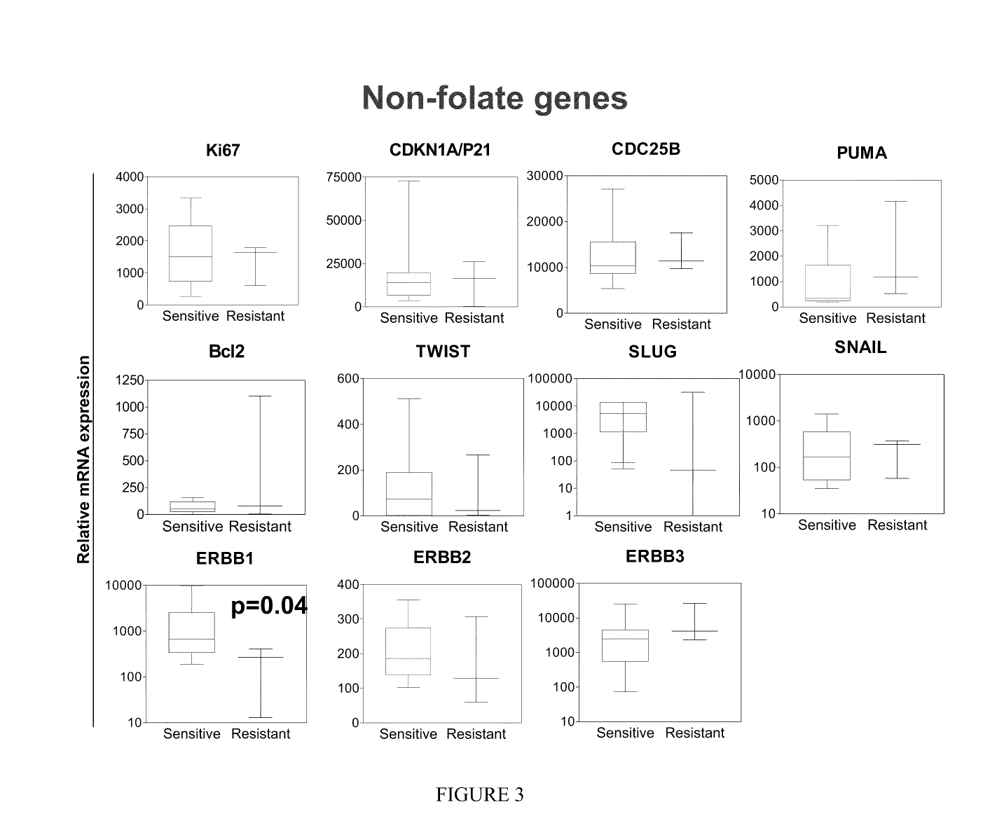

mRNA expression of genes encoding for cell cycle-related (Ki67, CDKN1/P21, CDC25A), apoptosis related (PUMA, Bcl2), EMT transcription factors (SLUG, SNAIL, TWIST) and tyrosine kinase receptors (ERBB1/EGFR, ERBB2, ERBB3) proteins was evaluated using quantitative RT-PCR (FIG. 3). The 10-propargyl-10-deazaminopterin sensitive cell lines were PC3, SCC61, DU145, HT29, HOP62, HOP92, SQ20B, HEP2, and IGROV1, with IC₅₀s ranging from 0.01 to 0.08 μM. The 10-propargyl-10-deazaminopterin resistant cell lines had IC₅₀s greater than 9 μM, and included MDA435, OVCAR3, HCT116, MCF7, HCC2998, and COL0205. No significant difference between “sensitive” and “resistant” groups was found for Ki67, CDKN1, CDC25A, PUMA, Bcl2, SLUG, SNAIL, TWIST, ERBB2 and ERBB3 expression. In contrast, cell lines with high ERBB1/EGFR expression were more sensitive to 10-propargyl-10-deazaminopterin than the cells expressing low level of this gene (t-test, p=0.04).

### Example 4

To further analyze the correlation between sensitivity to pralatrexate and increased EGFR expression the phosphorylation status of EGFR and some of its downstream signaling pathway components were analyzed by western blotting.

Western Blot Analysis.

Cells were lysed in buffer containing 50 mM HEPES (pH 7.6), 150 mM NaCl, 1% Triton X-100, 2 mM sodium vanadate, 100 mM NaF, and 0.4 mg/ml phenylmethylsulfonyl fluoride. Equal amounts of protein (20-50 μg/lane) were subjected to SDS-PAGE and transferred to nitrocellulose membranes. Membranes were blocked with 5% milk in 0.01% Tween 20/phosphate-buffered saline and then incubated with the primary antibody overnight. Membranes were then washed and incubated with the secondary antibody conjugated to horseradish peroxidase. Bands were visualized by using the enhanced chemiluminescence Western blotting detection system. Densitometric analysis was performed under conditions that yielded a linear response. The following antibodies were used: anti-cleaved PARP (Cell Signaling, Saint Quentin Yvelines, France), anti-DHFR (Abcam, France), anti-β-actin (Sigma Aldrich, Saint-Quentin Fallavier, France).

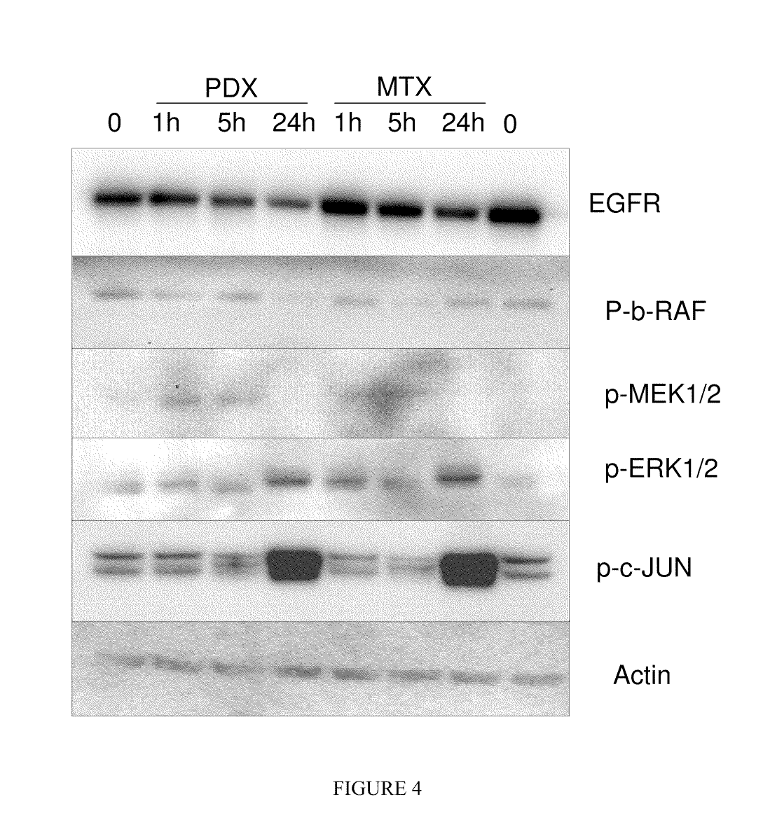

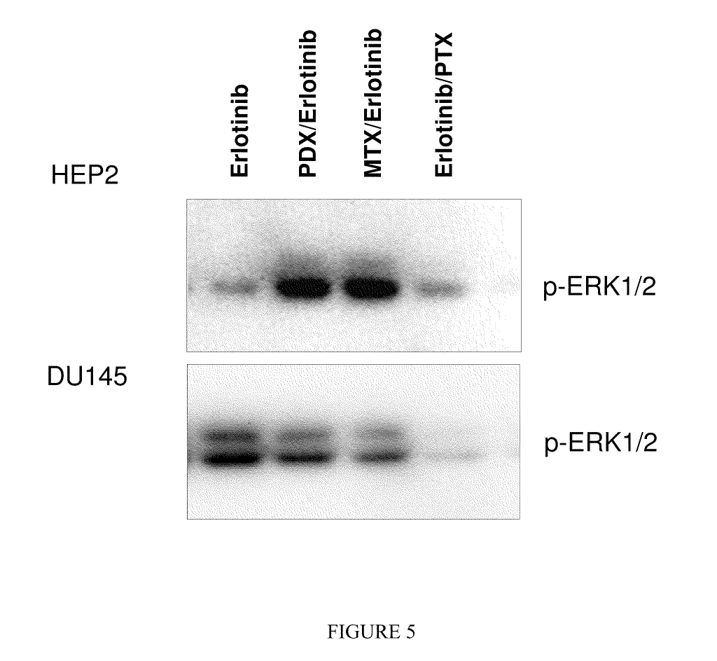

The correlation between pralatrexate sensitivity and EGFR expression led us to evaluate the phosphorylation status of EGFR and several downstream targets in 10-propargyl-10-deazaminopterin treated cells. We evaluated the effects of subtoxic concentrations of 10-propargyl-10-deazaminopterin and methotrexate on phosphorylation of several tyrosine kinases of EGFR/MAPK signaling pathway in HEP2 cells known to have high functional level of EGFR. As shown in FIG. 4, 10-propargyl-10-deazaminopterin and methotrexate had no effects on phosphorylation of b-RAF. 24 hour exposure to 10-propargyl-10-deazaminopterin and methotrexate induced activation of MEK1/2, ERK1/2 and c-JUN, suggesting activation of MAPK signaling pathway and transcription regulated by AP1 transcription factor. The basal level of EGFR protein was slightly diminished after 24 h exposure to 10-propargyl-10-deazaminopterin and methotrexate. We found that 10-propargyl-10-deazaminopterin treatment resulted in time-dependent phosphorylation of EGFR, ERK1/2, MEK1/2 and c-JUN, and this phosphorylation was abrogated by EGFR inhibitors. FIG. 5 shows effects of pralatrexate-erlotinib combination on phosphorylation of ERK 1-2.

### Example 5

This example describes testing of 10-propargyl-10-dAM and Tarceva® for cytotoxicity against human non-small cell lung cancer cell lines.

The objective of this study was to evaluate the effect of 10-propargyl-10-deazaminopterin alone and in combination with Tarceva® in the A549 non-small cell lung cancer xenograft mouse model. This study consisted of two parts. In the first part, the maximum tolerated dose (MTD) for 10-propargyl-10-deazaminopterin and Tarceva® alone and in combination was evaluated, with emphasis on the MTD of the combination. In the second part of the study, the effect of 10-propargyl-10-deazaminopterin alone and in combination with Tarceva® was evaluated in the A549 xenograft mouse model. Based on the MTD evaluation, nude mice tolerated 2 mg/kg dose of 10-propargyl-10-deazaminopterin (two cycles—QDx5) alone and in combination with 50 mg/kg of Tarceva® (QDx30). Results showed that 10-propargyl-10-deazaminopterin not only in combination with Tarceva® but also alone was effective in controlling A549 tumor growth.

Materials and Methods

Test System:

| | |
|---|---|
| Species/Strain: | Athymic nu/nu (HSD: ATHYMIC NUDE- |
| | FOXN1^{NU}) mice |
| Physiological state: | Immunodeficient mice |
| Age/weight range at | 4-6 week/15-18 gm |
| start of study: | |
| Sex: | Female |
| Animal supplier: | Harlan |
| Number of animals/group: | MTD evaluation - 5 animals/group |
| | Xenograft study - 10 animals/group |
| Identification: | Ear punch |
| Randomization: | Mean tumor volume in all groups should be |
| | within 15% of the mean tumor volume in |
| | Group 1 (control group) |
| Replacement: | Animals will not be replaced during the |
| | course of the study |

### Animal Housing and Environment:

| | |
|---|---|
| Housing: | Micro-isolator cages, 5 animals/cage |
| Acclimation: | 5 days |
| Environmental conditions: | Maintain under pathogen free environment |
| Food: | Irradiated certified standard fresh rodent |
| | chow |
| Water: | ad-libitum sterile water |

Test Articles:

| | | FORMULATION/ | |
|---|---|---|---|
| | | GENERAL | STORAGE |
| NAME | CODE or CAS | INFORMATION | CONDITIONS |
| PDX | | PBS | 4° C. |

### Control Article:

| | | FORMULATION/ | |
|---|---|---|---|
| | | GENERAL | STORAGE |
| NAME | CODE or CAS | INFORMATION | CONDITIONS |
| Tarceva ® | NDC# 50242-0063-01 | 0.5 CMC | 4° C. |

Description of the Cell Line Used for Xenotransplant

| | Cell | Histological | | |
|---|---|---|---|---|
| | Line | Origin | | |
| Tumor Type | (Name) | (Condition) | Growth media | Source |
| Non-small | A549 | Carcinoma | Ham's F12K medium | ATCC |
| cell lung | | | with 2 mM L- | |
| carcinoma | | | glutamine 1.5 g/L | |
| | | | sodium bicarbonate, | |
| | | | supplemented with 10% | |
| | | | fetal bovine serum | |

Methods:

Cell Lines and Cell Culture:

The human non small lung carcinoma cell line A549 was purchased from the American Type Culture Collection (ATCC, Manassas, Va.). The cell line was cultured in Ham's F 12K medium with 2 mM L-glutamine 1.5 g/L sodium bicarbonate, supplemented with 10% fetal bovine serum and was maintained at 37° C. in a humidified atmosphere at 5% CO₂ and 95% air.

Drug Preparations:

10-propargyl-10-deazaminopterin was prepared as described in U.S. Pat. No. 6,028,071 at a concentration of 20 mg/ml (PDX-008, lot #13110606). Dosing concentrations for 1 mg/kg and 2 mg/kg were prepared in PBS for IP dosing. Tarceva® was purchased from OSI Pharmaceuticals, Inc. (Melville, N.Y.) which is distributed by Genentech, Inc (CA). Tarceva® tablets were ground into powder, suspended in 0.5% Carboxymethyl cellulose (CMC) and dosed at 50 mg/kg by oral gavages.

Animal Studies:

Female athymic nu/nu mice, weighing approximately 18 g at 6-8 weeks of age, were obtained from Harlan Sprague Dawley, Inc (Indianapolis, Ind.). All mice were maintained in a laminar airflow cabinet under specific pathogen-free conditions. All facilities were approved by the Association of Assessment and Accreditation of Laboratory Animal Care (AALAC), and all animal experiments were conducted under the institutional guidelines established by the IACUC.

Evaluation of Maximum Tolerated Dose in Non-Tumor Bearing Nude Mice:

To evaluate the doses to be tested in the xenograft mouse model, non-tumor bearing female athymice nu/nu mice were treated with 2 mg/kg of 10-propargyl-10-deazaminopterin alone and in combination with 50 mg/kg of Tarceva® as described in Table 1. Animals were monitored daily for routine health observation. Body weight of the animals was recorded daily during the treatment phase and thereafter twice weekly for 14 days after the last treatment).

TABLE 1
Evaluation of maximum tolerated dose
(MTD in non-tumor bearing nude mice.

| | Number Of | | |
|---|---|---|---|
| Group | Animals Per | | |
| Number | Group | Description | Comments |
| 1 | 5 | PBS and | Route: IP/PO; PBC (QD × 5 for |
| | | 0.5% CMC | two cycles) plus 0.5% CMC |
| | | | (QD × 21) |
| 2 | 5 | PDX (2 mg/kg) | Route: IP; QD × 5 for |
| | | | two cycles |
| 3 | 5 | Tarceva (50 | Route: PO; QD |
| | | mg/kg) alone | |
| 4 | 5 | PDX (2 mg/kg) | Route: IP/PO; PDX (QD × 5 for |
| | | plus Tarceva | two cycles) plus Tarceva |
| | | (50 mg/kg) | (QD × 21) |

Establishment of Tumor Growth:

Xenotransplant of A549 was established by subcutaneous inoculation of 5×10⁶ cells on the right flank using a 21G needle in 6-8 week female athymic nude mice. A total of 75 animals were injected with A549 xenotransplant. Tumor volume was monitored twice weekly, once the established tumor reached 75-150 mm³ (individual tumor range between 70 to 200 mm³) the mice were randomized to the different groups as described in Table 2.

Tumor Measurements:

Tumor volume and body weights were measured twice weekly up to 58 days following A549 inoculation. Tumor volume was calculated using the following formula: Tumor volume=(A²×B/2) where A is the smallest diameter and B is the largest diameter.

Drug Treatment:

The treatment was initiated on the day of randomization according to the schedule described in Table 2. 10-propargyl-10-deazaminopterin was administered via IP injection and Tarceva® by oral gavage. PBS and 0.5% CMC were administrated to Group 1 as control vehicles by IP and PO routes, respectively.

TABLE 2
Dosing regimen of 10-propargyl-10-deazaminopterin
alone and in combination with Tarceva ® in
A549 xenograft mouse model

| | Number Of | | |
|---|---|---|---|
| Group | Animals Per | | |
| Number | Group | Description | Dosing/Schedule |
| 1 | 10 | Vehicle | PO (0.1 ml) |
| | | | OD |
| 1 | 10 | PDX (1 mg/kg) | IP (0.1 ml) |
| | | | QD × 5 for two cycles |
| 3 | 10 | PDX (2 mg/kg) | IP (0.1 ml) |
| | | | QD × 5 for two cycles |
| 4 | 10 | Tarceva (50 | PO (0.1 ml) |
| | | mg/kg) | OD |
| 5 | 10 | Tarceva/PDX | PO/IP (0.1 ml/0.1 ml) |
| | | 50 mg/kg/1 | OD/QD × 5 for two cycles |
| | | mg/kg | |
| 6 | 10 | Tarceva/PDX | PO/IP (0.1 ml/0.1 ml) |
| | | 50 mg/kg/2 | OD/QD × 5 for two cycles |
| | | mg/kg | |

Statistical Methods:

Statistical analysis was performed using Graphpad Prism software. Comparison between control group and treatment groups was performed using 2 way ANOVA analysis.

Results

Evaluation of 1050 in A549 Cells

An in vitro study in A549 cells was performed to compare the growth inhibition activity of 10-propargyl-10-deazaminopterin to that of cisplatin, paclitaxel, and docetaxel. The results are shown in Table 3.

TABLE 3

| Compound | IC50 (nM) |
|---|---|
| Pralatrexate | 65.1 |
| Cisplatin | 22.4 |
| Paclitaxel | 108.7 |
| Docetaxel | 23.6 |

Evaluation of Maximum Tolerated Dose in Non-Tumor Bearing Nude Mice:

In the MTD study, non-tumor bearing mice were exposed to 10-propargyl-10-deazaminopterin at 2 mg/kg of 10-propargyl-10-deazaminopterin (QDx5 for 2 cycles) alone and in combination with 50 mg/kg of Tarceva® (50 mg/kg (QDx21). Two of the five animals receiving 10-propargyl-10-deazaminopterin plus Tarceva® showed significant decrease in body weight over the period of 3 days at days 7, 8, and 9. These animals recovered the loss in body weight after day 9 of treatment. The maximum tolerated dose of the combination was determined as 10-propargyl-10-deazaminopterin 2 mg/kg+Tarceva® 50 mg/kg. Hence, a single dose of 10-propargyl-10-deazaminopterin or Tarceva® was well tolerated and the combination was acceptably tolerated in this study.

Evaluation of 10-propargyl-10-deazaminopterin Alone and in Combination with Tarceva® in A549 Xenograft Tumor Model:

Seventy five nude female mice were inoculated subcutaneously in the right flank with 5×10⁶ A549 cells to establish the xenotransplant. Tumor volume and body weights were monitored twice a week. Once the established tumor reached 75-150 mm³ (individual tumor range between 70 to 200 mm³) the mice were randomized to a treatment group as described in Table 2 above. The mean tumor volume in each of the treatment groups was within less than 1% that of mean tumor volume in the vehicle control group. Treatment was initiated on the day of randomization according to the schedule described in Table 2 above. One mouse each from group 5 and 6 was eliminated from the analysis due to ulceration of tumor. 10-propargyl-10-deazaminopterin was administered by IP injection and Tarceva® by oral gavage. Phosphate buffered saline and 0.5% carboxymethylcellulose were administered to Group 1 as control vehicles. 10-propargyl-10-deazaminopterin alone and in combination with Tarceva® showed better control of tumor growth, compared to the vehicle group, than the Tarceva® alone treated group. Interestingly, 10-propargyl-10-deazaminopterin at a dose of 2 mg/kg in combination with Tarceva® at 50 mg/kg showed significant tumor growth inhibition over the time period studied, compared to vehicle, 10-propargyl-10-deazaminopterin or Tarceva® alone groups. Also both the 10-propargyl-10-deazaminopterin alone groups (1 mg/kg and 2 mg/kg) and 10-propargyl-10-deazaminopterin (1 mg/kg) in combination with Tarceva® (50 mg/kg) showed a trend in reducing tumor growth, whereas Tarceva® alone at 50 mg/kg did not control tumor growth. Mean±SD tumor volume at the end of study (at 35^{th} day following start of therapy) was 386±244 mm³, 224±128 mm³, 210±109 mm³, 358±282 mm³, 243±122 mm³ and 152±85 mm³ in control, 10-propargyl-10-deazaminopterin (1 mg/kg), 10-propargyl-10-deazaminopterin (2 mg/kg), Tarceva® (50 mg/kg), 10-propargyl-10-deazaminopterin (1 mg/kg)+Tarceva® (50 mg/kg) and 10-propargyl-10-deazaminopterin (2 mg/kg)+Tarceva® (50 mg/kg), respectively.

Table 4 shows the effect of 10-propargyl-10-deazaminopterin/Tarceva® in A549 mouse model compared between control and treatment groups over the study period performed by 2-way ANOVA analysis using PRISM software.

TABLE 4

| | | | 10-propargyl-10- | | | | | | | | | |
|---|---|---|---|---|---|---|---|---|---|---|---|---|
| | | | deazaminopterin | | | | | | PDX (1 mg/kg) + | | PDX (2 mg/kg) + | |
| | Control | | (1 mg/kg) | | PDX (2 mg/kg) | | Tarceva (50 mg/kg) | | Tarceva (50 mg/kg) | | Tarceva (50 mg/kg) | |
| Days | Avg ± SD | P value* | Avg ± SD | P value | Avg ± SD | P value | Avg ± SD | P value | Avg ± SD | P value | Avg ± SD | P value |
| 0 | 132 ± 54. | — | 132 ± 52. | P > 0.05 | 131 ± 49. | P > 0.05 | 131 ± 47. | P > 0.05 | 131 ± 45. | P > 0.05 | 131 ± 42. | P > 0.05 |
| 3 | 190 ± 84. | — | 148 ± 67. | P > 0.05 | 133 ± 60. | P > 0.05 | 166 ± 80. | P > 0.05 | 167 ± 74. | P > 0.05 | 147 ± 73. | P > 0.05 |
| 7 | 223 ± 81. | — | 176 ± 87. | P > 0.05 | 166 ± 75. | P > 0.05 | 216 ± 121 | P > 0.05 | 160 ± 70. | P > 0.05 | 137 ± 56. | P > 0.05 |
| 10 | 219 ± 132 | — | 158 ± 93. | P > 0.05 | 139 ± 70. | P > 0.05 | 214 ± 140 | P > 0.05 | 159 ± 76. | P > 0.05 | 104 ± 66. | P > 0.05 |
| 14 | 256 ± 130 | — | 199 ± 79. | P > 0.05 | 207 ± 100 | P > 0.05 | 249 ± 130 | P > 0.05 | 214 ± 71. | P > 0.05 | 129 ± 67. | P > 0.05 |
| 17 | 289 ± 155 | — | 190 ± 78. | P > 0.05 | 174 ± 104 | P > 0.05 | 271 ± 206 | P > 0.05 | 204 ± 96. | P > 0.05 | 113 ± 61. | P < 0.05 |
| 21 | 323 ± 226 | — | 179 ± 112 | P > 0.05 | 194 ± 114 | P > 0.05 | 270 ± 210 | P > 0.05 | 201 ± 102 | P > 0.05 | 118 ± 40. | P < 0.01 |
| 24 | 361 ± 213 | — | 191 ± 105 | P < 0.05 | 194 ± 101 | P > 0.05 | 294 ± 261 | P > 0.05 | 224 ± 115 | P > 0.05 | 119 ± 83. | P < 0.001 |
| 28 | 366 ± 230 | — | 201 ± 114 | P > 0.05 | 189 ± 106 | P < 0.05 | 308 ± 280 | P > 0.05 | 223 ± 117 | P > 0.05 | 137 ± 80. | P < 0.01 |
| 35 | 386 ± 244 | — | 224 ± 128 | P > 0.05 | 210 ± 109 | P < 0.05 | 357 ± 282 | P > 0.05 | 243 ± 122 | P > 0.05 | 152 ± 85. | P < 0.01 |

*Comparison between control and treatment group over a study period was performed by 2-way ANOVA analysis using prism software.

In summary, this Example shows the effect of pralatrexate (10-propargyl-10-deazaminopterin) alone and in combination with Tarceva® on tumor growth inhibition in the A549 non-small cell lung cancer (NSCLC) cells and xenograft mouse model (female athymic nu/nu mice). An initial dose range finding study identified the maximum tolerated doses of 10-propargyl-10-deazaminopterin and Tarceva® alone and in combination in mice. In the tumor growth study, the effect of Tarceva® alone (50 mg/kg, PO, QDx30), 10-propargyl-10-deazaminopterin alone (1 mg/kg and 2 mg/kg, IP, two cycles—QDx5) or the combination of Tarceva® (50) and 10-propargyl-10-deazaminopterin (1 and 2) on subcutaneous A549 NSCLC growth was monitored for 35 days. 10-propargyl-10-deazaminopterin at 2 mg/kg in combination with Tarceva® at 50 mg/kg was significantly more effective in controlling A549 tumor growth, compared to control and Tarceva®-alone treated groups. 10-propargyl-10-deazaminopterin at 2 mg/kg in combination with Tarceva® was more effective in controlling tumor growth than either agent alone. 10-propargyl-10-deazaminopterin was shown to be effective at controlling tumor growth in this NSCLC xenograft model and may be a promising agent in the treatment of NSCLC.

### Example 6

This Example describes the differential activity and potential mechanism of action of pralatrexate (10-propargyl-10-deazaminopterin) methotrexate, and pemetrexed (Alimta) in human cancer models in vivo and in vitro.

Methods: This pilot study investigated activity and potential mechanism of action of 10-propargyl-10-deazaminopterin that differentiates it from other antifolates. We compared in vivo activity of 10-propargyl-10-deazaminopterin and MTX (both at 1 mg/kg and 2 mg/kg), Alimta (150 mg/kg) against MV522 and H460 non-small cell lung carcinoma human tumor xenografts. Endpoints included mean tumor growth inhibition or regression. We further evaluated activity and/or expression of folate-dependent enzymes: DHFR, thymidylate synthase (TS), and folylpolyglutamate synthase (FPGS) using kinetic assays and immunostaining in tumors harvested at the end of the study from animals treated with the antifolates or vehicle controls. In vitro studies included evaluation of reduced folate carrier (RFC) expression by quantitative RT-PCR in MV522, and H460, xenograft models. We also compared the short-term uptake (assessment of RFC-1 transporter) and on FPGS activity (polyglutamylation) of radiolabeled 10-propargyl-10-deazaminopterin, MTX (both at 5 μM) and Alimta (20 μM) in H460 cells in vitro. Inhibition of DHFR by the three antifolates was quantified in a cell-free assay against human recombinant DHFR.

Results and conclusions: In vivo, 10-propargyl-10-deazaminopterin dose-dependently inhibited tumor growth in H460 and MV522 xenografts with greater activity seen in the more rapidly growing H460 model. The activity of MTX and Alimta was also model-dependent, with 10-propargyl-10-deazaminopterin being more active than MTX or Alimta in each tumor xenograft model. RFC expression was below the assay detection limit in MV522 and H460 cells. 10-propargyl-10-deazaminopterin, Alimta and MTX exerted qualitative differences on TS enzymatic activity and protein expression, and DHFR protein content in H460 and MV522 tumor xenografts. Unlike Alimta and MTX, 10-propargyl-10-deazaminopterin down-regulated most of these endpoints.

Apparent Ki values for DHFR inhibition in a cell-free assay were, respectively, 45 nM, 26 nM, and >200 nM for 10-propargyl-10-deazaminopterin, MTX, and Alimta, respectively. The total uptake of radiolabeled drugs measured at 15 and 60 minutes was similar at both times (MTX), decreased later (Alimta), or increased over time (10-propargyl-10-deazaminopterin). A significantly smaller fraction of radiolabeled MTX entered the cells in comparison with 10-propargyl-10-deazaminopterin or Alimta. Radiolabeled species (conceivably polyglutamylated 10-propargyl-10-deazaminopterin) with a bell-shaped distribution of different Rf values than the drug alone appeared in a time-dependent manner in lysates from 10-propargyl-10-deazaminopterin-treated cells with much lesser polyglutamylation seen in MTX or Alimta-treated cells, suggesting greater polyglutamylation of 10-propargyl-10-deazaminopterin than either MTX or Alimta. The results from this pilot study suggest that 10-propargyl-10-deazaminopterin has a different activity profile relative to MTX and Alimta. Some of the observed differences include enhanced uptake of 10-propargyl-10-deazaminopterin into the cell and subsequent greater intracellular polyglutamylation that translate into greater tumor growth inhibition by 10-propargyl-10-deazaminopterin than either MTX or Alimta in NSCLC xenograft models in vivo.

### Example 7

To further explore a possible interaction between pralatrexate activity and the EGFR signaling pathway, combination treatments of pralatrexate and EGFR inhibitors (erlotinib and lapatinib) were investigated in the MTT assay.

Combination Treatments

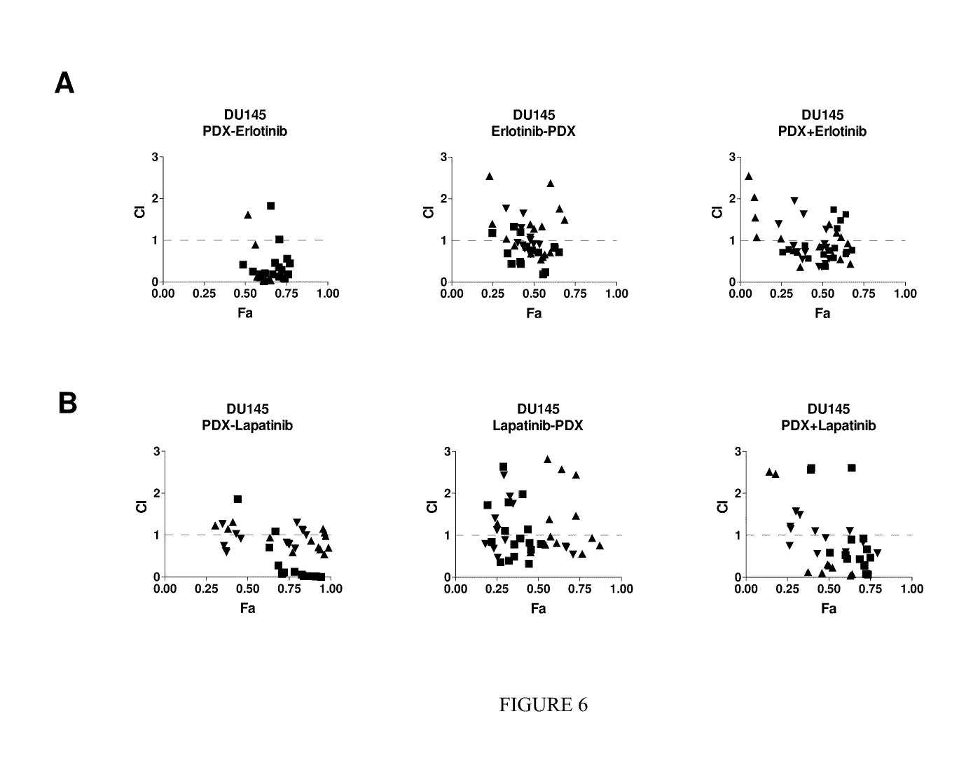

For combination studies of 10-propargyl-10-deazaminopterin with EGFR inhibitors, sequential or simultaneous schedules were implemented comprising: FIGS. 6A and 6B, Panel 1-sequential exposure to 10-propargyl-10-deazaminopterin (24 hours) followed by the EGFR inhibitor (24 hours); Panel 2-sequential exposure with the EGFR inhibitor (24 h) followed by treatment with 10-propargyl-10-deazaminopterin (for 24 h); Panel 3-simultaneous exposure to both agents (24 h). In sequential exposure schedules, cells were seeded and allowed to grow in the presence of various concentrations of 10-propargyl-10-deazaminopterin, erlotinib, or lapatinib for 24 or 48 h. The supernatant was then removed and the second compound was added. After an additional exposure period, the second compound was removed and cells were allowed to recover in drug-free medium for 72 h. Growth inhibition was then determined by the MTT assay. For simultaneous exposure, cells were seeded and treated after 24 hours with increasing concentrations of 10-propargyl-10-deazaminopterin alone or with the two other drugs in various concentrations corresponding to the IC₂₀, IC₄₀, IC₆₀ and IC₈₀ values. After approximately four doubling times (120 hours), the growth inhibitory effects were measured using the MTT assay.

Statistical Analysis and Determination of Synergistic Activity

Drug combination effects were determined using the Chou and Talalay method (Chou, 1984) based on the median effect principle. On the basis of the slope of the dose-effect curve for each different compound combination, it can be determined whether the compounds have mutually non-exclusive effects. Combination index (CI) values of <1 indicate synergy, a value of 1 indicates additive effects and a value of >1 indicates antagonism. Data were analyzed using the concentration-effect analysis software (Biosoft, Cambridge, UK). For statistical analysis and graphs, the Instat and Prism software (GraphPad, San Diego, USA) were used. Experiments were performed three times, in duplicate. Means and standard deviations were compared using Student's t-test (two-sided p value).

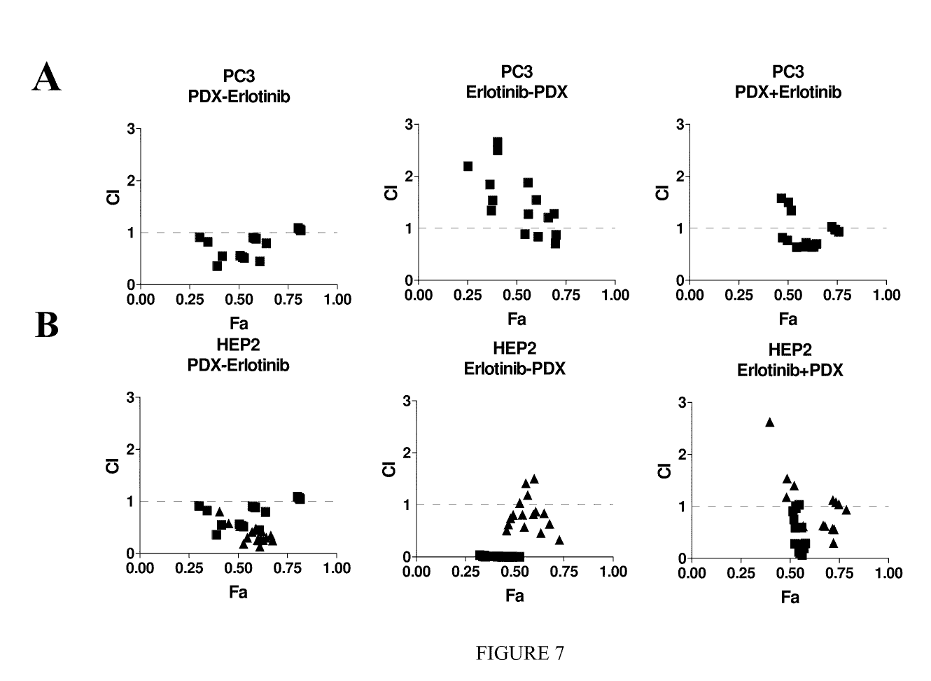

Pralatrexate was combined with two EGFR inhibitors, erlotinib and lapatinib (dual EGFR and HER2/Neu inhibitor) using sequential and simultaneous schedules of administration (FIGS. 6A and 6B). The combinations of pralatrexate with erlotinib and lapatinib in DU145 cells were shown to be synergistic when pralatrexate was given first or simultaneously with EGFR inhibitor, suggesting that inhibition of folate biosynthesis may activate EGFR survival pathway and facilitate its inhibition. Similar results were obtained using prostate PC3 and head and neck HEP2 cancer cell lines (FIGS. 7A and 7B.).

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method of selecting a patient for treatment of a cancer with 10-propargyl-10-deazaminopterin, the method comprising the steps of:
(a) obtaining a sample of the patient's cancer tissue;
(b) determining the expression level of epidermal growth factor receptor (EGFR) expressed in the sample;
(c) comparing the determined expression level in the sample with a reference expression level for EGFR; and
(d) selecting the patient for treatment 10-propargyl-10-deazaminopterin where the comparison of the expression level in the sample of EGFR and the corresponding reference expression level indicate sensitivity of patient's cancer tissue to 10-propargyl-10-deazaminopterin.

2. The method of claim 1, wherein the patient's cancer is a solid tumor or a lymphoma.

3. The method of claim 2, wherein the solid tumor is selected from the group consisting of NSCLC, head and neck cancer, prostate cancer, and breast cancer.

4. A method for assessing sensitivity of a patient's cancer to treatment with 10-propargyl-10-deazaminopterin comprising the steps of:
(a) obtaining a sample of the patient's cancer tissue;
(b) determining the expression level of at EGFR expressed in the sample;
(c) comparing the determined expression level in the sample with a reference expression level for EGFR to determine whether the expression level for EGFR in the sample is indicative of an expression level that predicts sensitivity to 10-propargyl-10-deazaminopterin; and
(d) generating a report of the predicted sensitivity of the sample to 10-propargyl-10-deazaminopterin.

5. The method of claim 4, wherein the reference cancer is a solid tumor or a lymphoma.

6. The method of claim 5, wherein the solid tumor is selected from the group consisting of NSCLC, head and neck cancer, prostate cancer, and breast cancer.

7. A method for assessing sensitivity of a cancer to treatment with 10-propargyl-10-deazaminopterin comprising the steps of
(a) obtaining a sample of the lymphoma;
(b) determining the amount of EGFR expressed by the sample wherein higher levels of expressed EGFR are indicative of sensitivity to 10-propargyl-10-deazaminopterin; and
(c) generating a report of the predicted sensitivity of the sample to 10-propargyl-10-deazaminopterin.
